(19) United States
(12) Patent Application Publication
(10) Pub. No.: US 2017/0233382 A1
(43) Pub. Date: Aug. 17, 2017

(54) PROCESSES FOR THE PREPARATION OF A BACE INHIBITOR

(71) Applicants: Steven P. MILLER, Rahway, NJ (US); Carmela MOLINARO, Rahway, NJ (US); David THAISRIVONGS, Rahway, NJ (US); Feng XU, Rahway, NJ (US); Richard DESMOND, Rahway (US); Hongming LI, Rahway, NJ (US); Qinghao CHEN, Rahway, NJ (US); Andrew STAMFORD, Rahway, NJ (US); Zhiguo Jake SONG, Rahway, NJ (US); Lushi TAN, Rahway, NJ (US); Merck Sharp & Dohme Corp., Rahway, NJ (US)

(72) Inventors: Steven P. Miller, Monroe, NJ (US); Carmela Molinaro, Colonia, NJ (US); David Thaisrivongs, Cranford, NJ (US); Feng Xu, Staten Island, NY (US); Richard Desmond, Lebanon, NJ (US); Hongming Li, Westfield, NJ (US); Qinghao Chen, Edison, NJ (US); Andrew W. Stamford, Chatham, NJ (US); Zhiguo Jake Song, Edison, NJ (US); Lushi Tan, Edison, NJ (US)

(73) Assignee: Merck Sharp & Dohme Corp., Rahway, NJ (US)

(21) Appl. No.: 15/503,035

(22) PCT Filed: Aug. 10, 2015
(86) PCT No.: PCT/US2015/044410

§ 371 Date: Feb. 10, 2017

Related U.S. Application Data
(60) Provisional application No. 62/037,423, filed on Aug. 14, 2014.
(60) Provisional application No. 62/182,117, filed on Jun. 19, 2015.

(51) Int. Cl. C07D 417/12; C07C 313/06; C07C 311/33; C07D 213/81

(52) U.S. Cl. CPC C07D 417/12; C07D 213/81; C07C 313/06; C07C 311/33

(57) ABSTRACT

This invention provides processes for the preparation of verubecestat (Compound of Formula (I)), a potent inhibitor of BACE-1 and BACE-2. In addition, the invention provides certain synthetic intermediates which are useful, among other things, for the preparation of the Compound of Formula (I).

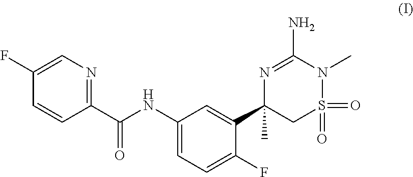 [SMILES: CN1C(N)=N[C@](C)(c2cc(NC(=O)c3ccc(F)cn3)ccc2F)CS1(=O)=O]

### FIELD OF THE INVENTION

This invention provides processes for the preparation of verubecestat (Compound of Formula (I)), a potent inhibitor of BACE-1 and BACE-2. In addition, the invention provides certain synthetic intermediates which are useful, among other things, for the preparation of the Compound of Formula (I).

### BACKGROUND

Amyloid beta peptide (“Aβ”) is a primary component of β amyloid fibrils and plaques, which are regarded as having a role in an increasing number of pathologies. Examples of such pathologies include, but are not limited to, Alzheimer's disease, Down's syndrome, Parkinson's disease, memory loss (including memory loss associated with Alzheimer's disease and Parkinson's disease), attention deficit symptoms (including attention deficit symptoms associated with Alzheimer's disease (“AD”), Parkinson's disease, and Down's syndrome), dementia (including pre-senile dementia, senile dementia, dementia associated with Alzheimer's disease, Parkinson's disease, and Down's syndrome), progressive supranuclear palsy, cortical basal degeneration, neurodegeneration, olfactory impairment (including olfactory impairment associated with Alzheimer's disease, Parkinson's disease, and Down's syndrome), β-amyloid angiopathy (including cerebral amyloid angiopathy), hereditary cerebral hemorrhage, mild cognitive impairment (“MCI”), glaucoma, amyloidosis, type II diabetes, hemodialysis ((β2 microglobulins and complications arising therefrom), neurodegenerative diseases such as scrapie, bovine spongiform encephalitis, Creutzfeld-Jakob disease, traumatic brain injury and the like.

Aβ peptides are short peptides which are made from the proteolytic break-down of the transmembrane protein called amyloid precursor protein (“APP”). Aβ peptides are made from the cleavage of APP by β-secretase activity at a position corresponding to the N-terminus of Aβ to produce a membrane-bound fragment C99. Gamma-secretase activity cleaves C99 at a position corresponding to the Aβ C-terminus of to produce Aβ. (APP is also cleaved by α-secretase activity, resulting in the secreted, non-amyloidogenic fragment known as soluble APPα). Beta site APP Cleaving Enzyme (“BACE-1”) is regarded as the primary aspartyl protease responsible for the production of Aβ by β-secretase activity. The inhibition of BACE-1 has been shown to inhibit the production of Aβ.

AD is estimated to afflict more than 20 million people worldwide and is believed to be the most common cause of dementia. AD is a disease characterized by degeneration and loss of neurons and also by the formation of senile plaques and neurofibrillary tangles. Presently, treatment of Alzheimer's disease is limited to the treatment of its symptoms rather than the underlying causes. Symptom-improving agents approved for this purpose include, for example, N-methyl-D-aspartate receptor antagonists such as memantine (Namenda®, Forest Pharmaceuticals, Inc.), cholinesterase inhibitors such as donepezil (Aricept®, Pfizer), rivastigmine (Exelon®, Novartis), galantamine (Razadyne Reminyl®), and tacrine (Cognex®).

In AD, Aβ peptides, formed through β-secretase and gamma-secretase activity, can form tertiary structures that aggregate to form amyloid fibrils. Aβ peptides have also been shown to form Aβ oligomers (sometimes referred to as “Aβ aggregates” or “Abeta oligomers”). Aβ oligomers are small multimeric structures composed of 2 to 12 Aβ peptides that are structurally distinct from Aβ fibrils. Amyloid fibrils can deposit outside neurons in dense formations known as senile plaques, neuritic plaques, or diffuse plaques in regions of the brain important to memory and cognition. Aβ oligomers are cytotoxic when injected in the brains of rats or in cell culture. This Aβ plaque formation and deposition and/or Aβ oligomer formation, and the resultant neuronal death and cognitive impairment, are among the hallmarks of AD pathophysiology. Other hallmarks of AD pathophysiology include intracellular neurofibrillary tangles comprised of abnormally phosphorylated tau protein, and neuroinflammation.

Evidence suggests that Aβ, Aβ fibrils, aggregates, oligomers, and/or plaque play a causal role in AD pathophysiology. (Ohno et al., Neurobiology of Disease, No. 26 (2007), 134-145). Mutations in the genes for APP and presenilins 1/2 (PS1/2) are known to cause familial AD and an increase in the production of the 42-amino acid form of Aβ is regarded as causative. Aβ has been shown to be neurotoxic in culture and in vivo. For example, when injected into the brains of aged primates, fibrillar Aβ causes neuronal cell death around the injection site. Other direct and circumstantial evidence of the role of Aβ in Alzheimer etiology has also been published.

BACE-1 has become an accepted therapeutic target for the treatment of Alzheimer's disease. For example, McConlogue et al., J. Bio. Chem., Vol. 282, No. 36 (September 2007), have shown that partial reductions of BACE-1 enzyme activity and concomitant reductions of Aβ levels lead to a dramatic inhibition of Aβ-driven AD-like pathology, making β-secretase a target for therapeutic intervention in AD. Ohno et al. Neurobiology of Disease, No. 26 (2007), 134-145, report that genetic deletion of BACE-1 in SXFAD mice abrogates Aβ generation, blocks amyloid deposition, prevents neuron loss found in the cerebral cortex and subiculum (brain regions manifesting the most severe amyloidosis in SXFAD mice), and rescues memory deficits in SXFAD mice. The group also reports that Aβ is ultimately responsible for neuron death in AD and concludes that BACE-1 inhibition has been validated as an approach for the treatment of AD. Roberds et al., Human Mol. Genetics, 2001, Vol. 10, No. 12, 1317-1324, established that inhibition or loss of β-secretase activity produces no profound phenotypic defects while inducing a concomitant reduction in Aβ. Luo et al., Nature Neuroscience, Vol. 4, No. 3, March 2001, report that mice deficient in BACE-1 have normal phenotype and abolished β-amyloid generation.

More recently, Jonsson, et al. have reported in Nature, Vol. 488, pp. 96-99 (August 2012), that a coding mutation (A673T) in aPP gene protects against Alzheimer's disease and cognitive decline in the elderly without Alzheimer's disease. More specifically, a allele of rs63750847, a single nucleotide polymorphism (SNP), results in an alanine to threonine substitution at position 673 in APP (A673T). This SNP was found to be significantly more common in a healthy elderly control group than in an Alzheimer's disease group. A673T substitution is adjacent to aspartyl protease beta-site in APP, and results in an approximately 40% reduction in the formation of amyloidogenic peptides in a heterologous cell expression system in vitro. Jonsson, et al. report that an APP-derived peptide substrate containing a673T mutation is processed 50% less efficiently by purified human BACE-1 enzyme when compared to a wild-type peptide. Jonsson et al. indicate that the strong protective effect of aPP-A673T substitution against Alzheimer's disease provides proof of principle for the hypothesis that reducing the beta-cleavage of APP may protect against the disease.

BACE-1 has also been identified or implicated as a therapeutic target for a number of other diverse pathologies in which Aβ or Aβ fragments have been identified to play a causative role. One such example is in the treatment of AD-type symptoms of patients with Down's syndrome. The gene encoding APP is found on chromosome 21, which is also the chromosome found as an extra copy in Down's syndrome. Down's syndrome patients tend to acquire AD at an early age, with almost all those over 40 years of age showing Alzheimer's-type pathology. This is thought to be due to the extra copy of aPP gene found in these patients, which leads to overexpression of APP and therefore to increased levels of Aβ causing the prevalence of AD seen in this population. Furthermore, Down's patients who have a duplication of a small region of chromosome 21 that does not include aPP gene do not develop AD pathology. Thus, it is thought that inhibitors of BACE-1 could be useful in reducing Alzheimer's type pathology in Down's syndrome patients.

Another example is in the treatment of glaucoma (Guo et al., PNAS, Vol. 104, No. 33, Aug. 14, 2007). Glaucoma is a retinal disease of the eye and a major cause of irreversible blindness worldwide. Guo et al. report that Aβ colocalizes with apoptotic retinal ganglion cells (RGCs) in experimental glaucoma and induces significant RGC cell loss in vivo in a dose- and time-dependent manner. The group report having demonstrated that targeting different components of aβ formation and aggregation pathway, including inhibition of β-secretase alone and together with other approaches, can effectively reduce glaucomatous RGC apoptosis in vivo. Thus, the reduction of Aβ production by the inhibition of BACE-1 could be useful, alone or in combination with other approaches, for the treatment of glaucoma.

Another example is in the treatment of olfactory impairment. Getchell et al., Neurobiology of Aging, 24 (2003), 663-673, have observed that the olfactory epithelium, a neuroepithelium that lines the posterior-dorsal region of the nasal cavity, exhibits many of the same pathological changes found in the brains of AD patients, including deposits of Aβ, the presence of hyperphosphorylated tau protein, and dystrophic neurites among others. Other evidence in this connection has been reported by Bacon A W, et al., Ann NY Acad Sci 2002; 855:723-31; Crino P B, Martin J A, Hill W D, et al., Ann Otol Rhinol Laryngol, 1995; 104:655-61; Davies D C, et al., Neurobiol Aging, 1993; 14:353-7; Devanand D P, et al., Am J Psychiatr, 2000; 157:1399-405; and Doty R L, et al., Brain Res Bull, 1987; 18:597-600. It is reasonable to suggest that addressing such changes by reduction of Aβ by inhibition of BACE-1 could help to restore olfactory sensitivity in patients with AD.

For compounds which are inhibitors of BACE-2, another example is in the treatment of type-II diabetes, including diabetes associated with amyloidogenesis. BACE-2 is expressed in the pancreas. BACE-2 immunoreactivity has been reported in secretory granules of beta cells, co-stored with insulin and IAPP, but lacking in the other endocrine and exocrine cell types. Stoffel et al., WO2010/063718, disclose the use of BACE-2 inhibitors in the treatment of metabolic diseases such as Type-II diabetes. The presence of BACE-2 in secretory granules of beta cells suggests that it may play a role in diabetes-associated amyloidogenesis. (Finzi, G. Franzi, et al., Ultrastruct Pathol. 2008 November-December; 32(6):246-51.)

Other diverse pathologies characterized by the formation and deposition of Aβ or fragments thereof, and/or by the presence of amyloid fibrils, oligomers, and/or plaques, include neurodegenerative diseases such as scrapie, bovine spongiform encephalitis, traumatic brain injury (“TBI”), Creutzfeld-Jakob disease and the like, type II diabetes (which is characterized by the localized accumulation of cytotoxic amyloid fibrils in the insulin producing cells of the pancreas), and amyloid angiopathy. In this regard reference can be made to the patent literature. For example, Kong et al., US2008/0015180, disclose methods and compositions for treating amyloidosis with agents that inhibit Aβ peptide formation. As another example, Loane, et al. report the targeting of amyloid precursor protein secretases as therapeutic targets for traumatic brain injury. (Loane et al., “Amyloid precursor protein secretases as therapeutic targets for traumatic brain injury”, Nature Medicine, Advance Online Publication, published online Mar. 15, 2009.) Still other diverse pathologies characterized by the inappropriate formation and deposition of Aβ or fragments thereof, and/or by the presence of amyloid fibrils, and/or for which inhibitor(s) of BACE are expected to be of therapeutic value are discussed further hereinbelow.

The compound:

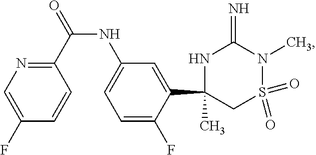 [SMILES: CN1C(=N)N[C@](C)(c2cc(NC(=O)c3ccc(F)cn3)ccc2F)CS1(=O)=O]

and its tautomer:

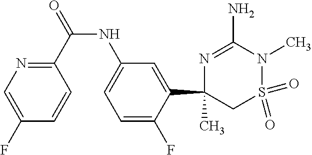 [SMILES: CN1C(N)=N[C@](C)(c2cc(NC(=O)c3ccc(F)cn3)ccc2F)CS1(=O)=O]

which are collectively and individually referred to herein as “verubecestat”, or, alternatively, as the “Compound of the Formula (I)”, and pharmaceutically acceptable salts thereof, are disclosed in U.S. Pat. No. 8,729,071, PCT Patent Publication No. WO2011/044181, and elsewhere as an inhibitor of BACE-1 and BACE-2, together with pharmaceutical compositions thereof, for use in treating, preventing, ameliorating, and/or delaying the onset of an Aβ pathology and/or a symptom or symptoms thereof, including Alzheimer's disease. A preparation of the Compound of Formula (I) is also disclosed therein.

The “endo” (or “amine”) tautomer of the Compound of Formula (I), which is shown above, may be depicted as

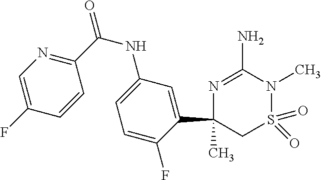 [SMILES: CN1C(N)=N[C@](C)(c2cc(NC(=O)c3ccc(F)cn3)ccc2F)CS1(=O)=O]

and named in the CAS style as N-[3-[(5R)-3-amino-5,6-dihydro-2,5-dimethyl-1,1-dioxido-2H-1,2,4-thiadiazin-5-yl]-4-fluorophenyl]-5-fluoro-2-pyridinecarboxamide, and in the IUPAC style as N-{3-[(5R)-3-amino-2,5-dimethyl-1,1-dioxo-5,6-dihydro-2H-Iλ⁶,2,4-thiadiazin-5-yl]-4-fluorophenyl}-5-fluoropyridine-2-carboxamide.

The “exo” (or “imine”) tautomer of the Compound of Formula (I), which is also shown above, may be depicted as

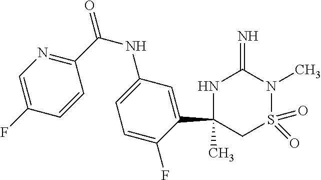 [SMILES: CN1C(=N)N[C@](C)(c2cc(NC(=O)c3ccc(F)cn3)ccc2F)CS1(=O)=O]

and named in the CAS style as 5-fluoro-N-[4-fluoro-3-[(5R)-tetrahydro-3-imino-2,5-dimethyl-1,1-dioxido-2H-1,2,4-thiadiazin-5-yl]phenyl]-2-pyridinecarboxamide, and in the IUPAC style as 5-Fluoro-N-{4-fluoro-3-[(5R)-3-imino-2,5-dimethyl-1,1-dioxo-Iλ⁶,2,4-thiadiazinan-5-yl]phenyl}pyridine-2-carboxamide.

U.S. Pat. No. 8,729,071 discloses preparation of the Compound of Formula (I) as Example 25 in Table V through coupling of an appropriate aryl amine and carboxylic acid. While the procedures disclosed therein are suitable for preparing working quantities of the Compound of Formula (I), alternative synthetic procedures for the preparation of the compound which are more amenable to scale-up are desirable.

### SUMMARY OF THE INVENTION

The present invention provides processes for the preparation of verubecestat (Compound of Formula (I)) which may be useful (alone or together with additional active ingredients) in treating, preventing, ameliorating, and/or delaying the onset of an Aβ pathology and/or a symptom or symptoms thereof. In addition, the invention provides certain synthetic intermediates which are useful, among other things, for the preparation of the Compound of Formula (I).

### DETAILED DESCRIPTION OF THE INVENTION

### Definitions

The terms used herein have their ordinary meaning and the meaning of such terms is independent at each occurrence thereof. That notwithstanding and except where stated otherwise, the following definitions apply throughout the specification and claims. Chemical names, common names, and chemical structures may be used interchangeably to describe the same structure. If a chemical compound is referred to using both a chemical structure and a chemical name, and an ambiguity exists between the structure and the name, the structure predominates. These definitions apply regardless of whether a term is used by itself or in combination with other terms, unless otherwise indicated. Hence, the definition of “alkyl” applies to “alkyl” as well as the “alkyl” portions of “hydroxyalkyl,” “haloalkyl,” “—O-alkyl,” etc.

As used herein, and throughout this disclosure, the following terms, unless otherwise indicated, shall be understood to have the following meanings:

The term “alkyl,” as used herein, refers to an aliphatic hydrocarbon group having one of its hydrogen atoms replaced with a bond having the specified number of carbon atoms. In different embodiments, an alkyl group contains from 1 to 6 carbon atoms (C₁-C₆ alkyl) or from 1 to 3 carbon atoms (C₁-C₃ alkyl). Non-limiting examples of alkyl groups include methyl, ethyl, n-propyl, isopropyl, n-butyl, sec-butyl, isobutyl, tert-butyl, n-pentyl, neopentyl, isopentyl, n-hexyl, isohexyl and neohexyl. In one embodiment, an alkyl group is linear. In another embodiment, an alkyl group is branched.

The term “haloalkyl,” as used herein, refers to an alkyl group as defined above, wherein one or more of the alkyl group's hydrogen atoms has been replaced with a halo. In one embodiment, a haloalkyl group has from 1 to 6 carbon atoms. In another embodiment, a haloalkyl group has from 1 to 3 carbon atoms. In another embodiment, a haloalkyl group is substituted with from 1 to 3 halo atoms. Non-limiting examples of haloalkyl groups include —CH₂F, —CHF₂, and —CF₃. The term “C₁-C₄ haloalkyl” refers to a haloalkyl group having from 1 to 4 carbon atoms.

The term “alkoxy” as used herein, refers to an —O-alkyl group, wherein an alkyl group is as defined above. Non-limiting examples of alkoxy groups include methoxy, ethoxy, n-propoxy, isopropoxy, n-butoxy and t-butoxy. An alkoxy group is bonded via its oxygen atom to the rest of the molecule.

The term “aryl,” as used herein, refers to an aromatic monocyclic or multicyclic ring system comprising from about 6 to about 14 carbon atoms. In one embodiment, an aryl group contains from about 6 to 10 carbon atoms (C₆-C₁₀ aryl). In another embodiment an aryl group is phenyl. Non-limiting examples of aryl groups include phenyl and naphthyl.

The term “cyanating agent,” as used herein, refers to an electrophilic agent suitable for transferring a cyano group to a nucleophilic reactant. Non-limiting examples of cyanating agents include cyanogen bromide, cyanogen fluoride, cyanogen chloride, cyanogen iodide, 2-methoxyphenyl cyanate, 4-methoxyphenyl cyanate, 4-phenylphenyl cyanate, and bisphenol A cyanate.

The term “Brønsted base,” as used herein, refers to an agent that accepts hydrogen ions during a chemical reaction. Non-limiting examples of Brønsted bases include potassium carbonate, potassium phosphate, cesium carbonate, and potassium bicarbonate.

The term “PG”, as used herein in text and in structural depictions of certain compounds herein (e.g., compounds 4, 4A, 6, and 6A), refers to a protecting group. Those skilled in the art will readily envisage protecting groups (PG) suitable for use in the compounds and processes according to the invention. Protecting groups suitable for use herein include acid-labile protecting groups. Non-limiting examples of PG suitable for use herein include —S(O)₂R⁸, —C(O)OR⁸, —C(O)R⁸, —CH₂OCH₂CH₂SiR⁸, and —CH₂R₈ where R⁸ is selected from the group consisting of —C₁₋₈ alkyl (straight or branched), —C₃₋₈ cycloalkyl, —CH₂(aryl), and —CH(aryl)₂, wherein each aryl is independently phenyl or naphthyl and each said aryl is optionally independently unsubstituted or substituted with one or more (e.g., 1, 2, or 3) groups independently selected from —OMe, Cl, Br, and I. Preferred protecting groups “PG” include butoxycarbonyl (Boc) and para-methoxybenzyl (PMB).

The term “diazonium group,” as used herein, refers to a the functional group

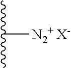

where

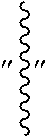

indicates the point of attachment to the parent group and X⁻ is an inorganic or organic anion such as a halide.

The term “halo,” as used herein, means —F (fluorine), —Cl (chlorine), —Br (bromine) or —I (iodine).

The term “substituted” means that one or more hydrogens on the atoms of the designated moiety are replaced with a selection from the indicated group, provided that the atoms' normal valencies under the existing circumstances are not exceeded, and that the substitution results in a stable compound. Combinations of substituents and/or variables are permissible only if such combinations result in stable compounds. By “stable compound′ or “stable structure” is meant a compound that is sufficiently robust to survive isolation to a useful degree of purity from a reaction mixture, and formulation into an efficacious therapeutic agent.

When any substituent or variable occurs more than one time in any constituent or the compound of Formula (I), its definition on each occurrence is independent of its definition at every other occurrence, unless otherwise indicated. For example, description of radicals which include the expression “—N(C₁-C₃ alkyl)₂” means —N(CH₃)(CH₂CH₃), —N(CH₃)(CH₂CH₂CH₃), and —N(CH₂CH₃)(CH₂CH₂CH₃), as well as —N(CH₃)₂, —N(CH₂CH₃)₂, and —N(CH₂CH₂CH₃)₂.

It should also be noted that any carbon as well as heteroatom with unsatisfied valences in the text, schemes, examples and tables herein is assumed to have the sufficient number of hydrogen atom(s) to satisfy the valences.

One or more compounds of the invention may exist in unsolvated as well as solvated forms with pharmaceutically acceptable solvents such as water, ethanol, and the like, and it is intended that the invention embrace both solvated and unsolvated forms. “Solvate” means a physical association of a compound of this invention with one or more solvent molecules. This physical association involves varying degrees of ionic and covalent bonding, including hydrogen bonding. In certain instances the solvate will be capable of isolation, for example when one or more solvent molecules are incorporated in the crystal lattice of the crystalline solid. “Solvate” encompasses both solution-phase and isolatable solvates. Non-limiting examples of suitable solvates include ethanolates, methanolates, and the like. “Hydrate” is a solvate wherein the solvent molecule is H₂O.

The compounds of Formula (I) may contain one or more stereogenic centers and can thus occur as racemates, racemic mixtures, single enantiomers, diastereomeric mixtures and individual diastereomers. Additional asymmetric centers may be present depending upon the nature of the various substituents on the molecule. Each such asymmetric center will independently produce two optical isomers and it is intended that all of the possible optical isomers and diastereomers in mixtures and as pure or partially purified compounds are included within the ambit of this invention. Any formulas, structures or names of compounds described in this specification that do not specify a particular stereochemistry are meant to encompass any and all existing isomers as described above and mixtures thereof in any proportion. When stereochemistry is specified, the invention is meant to encompass that particular isomer in pure form or as part of a mixture with other isomers in any proportion.

Diastereomeric mixtures can be separated into their individual diastereomers on the basis of their physical chemical differences by methods well known to those skilled in the art, such as, for example, by chromatography and/or fractional crystallization. Enantiomers can be separated by converting the enantiomeric mixture into a diastereomeric mixture by reaction with an appropriate optically active compound (e.g., chiral auxiliary such as a chiral alcohol or Mosher's acid chloride), separating the diastereomers and converting (e.g., hydrolyzing) the individual diastereomers to the corresponding pure enantiomers. Also, some of the compounds of Formula (I) may be atropisomers (e.g., substituted biaryls) and are considered as part of this invention. Enantiomers can also be separated by use of chiral HPLC column.

As noted above, verubecestat, alternatively referred to herein as the “Compound of Formula (I),” may exist as either of two tautomeric forms: the “exo” (or “imine”) form and the “endo” (or “amine”) form, which are shown above. For ease of description, and unless otherwise specified, the expression “a Compound of Formula (I):

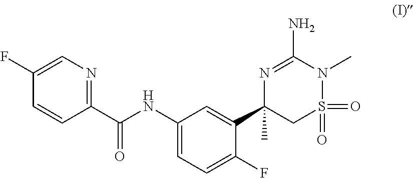 [SMILES: CN1C(N)=N[C@](C)(c2cc(NC(=O)c3ccc(F)cn3)ccc2F)CS1(=O)=O]

is intended to encompass the endo, or the exo form, or a mixture of both of the endo and exo tautomeric forms.

All stereoisomers (for example, geometric isomers, optical isomers and the like) of the present compounds (including those of the salts and solvates of the compounds as well as the salts, solvates and esters of the prodrugs), such as those which may exist due to asymmetric carbons on various substituents, including enantiomeric forms (which may exist even in the absence of asymmetric carbons), rotameric forms, atropisomers, and diastereomeric forms, are contemplated within the scope of this invention. Individual stereoisomers of the compounds of the invention may, for example, be substantially free of other isomers, or may be admixed, for example, as racemates or with all other, or other selected, stereoisomers. The chiral centers of the present invention can have the S or R configuration as defined by the IUPAC 1974 Recommendations.

The compounds of Formula (I) and intermediates for the preparation thereof can form salts which are also within the scope of this invention. Reference to a compound of Formula (I) or synthetic intermediate useful for its prepation herein is understood to include reference to salts thereof, unless otherwise indicated. The term “salt(s)”, as employed herein, denotes acidic salts formed with inorganic and/or organic acids, as well as basic salts formed with inorganic and/or organic bases. In addition, when a compound of Formula (I) or a synthetic intermediate contains both a basic moiety, such as, but not limited to a pyridine or imidazole, and an acidic moiety, such as, but not limited to a carboxylic acid, zwitterions (“inner salts”) may be formed and are included within the term “salt(s)” as used herein. Such acidic and basic salts used within the scope of the invention are pharmaceutically acceptable (i.e., non-toxic, physiologically acceptable) salts. Salts of the compounds of Formula (I) or a synthetic intermediate may be formed, for example, by reacting a compound of Formula (I) (or synthetic intermediate) with an amount of acid or base, such as an equivalent amount, in a medium such as one in which the salt precipitates or in an aqueous medium followed by lyophilization.

Exemplary acid addition salts include acetates, ascorbates, benzoates, benzenesulfonates, bisulfates, borates, butyrates, citrates, camphorates, camphorsulfonates, fumarates, hydrochlorides, hydrobromides, hydroiodides, lactates, maleates, methanesulfonates, naphthalenesulfonates, nitrates, oxalates, phosphates, propionates, salicylates, succinates, sulfates, tartrates, thiocyanates, toluenesulfonates (also known as tosylates), 1-hydroxy-2-naphthoates (also known as xinafoates) and the like. Additionally, acids which are generally considered suitable for the formation of pharmaceutically useful salts from basic pharmaceutical compounds are discussed, for example, by P. Stahl et al, Camille G. (eds.) Handbook of Pharmaceutical Salts. Properties, Selection and Use. (2002) Zurich: Wiley-VCH; S. Berge et al, Journal of Pharmaceutical Sciences (1977) 66(1) 1-19; P. Gould, International J. of Pharmaceutics (1986) 33 201-217; Anderson et al, The Practice of Medicinal Chemistry (1996), Academic Press, New York; and in The Orange Book (Food & Drug Administration, Washington, D.C. on their website). These disclosures are incorporated herein by reference.

Exemplary basic salts include ammonium salts, alkali metal salts such as sodium, lithium, and potassium salts, alkaline earth metal salts such as calcium and magnesium salts, salts with organic bases (for example, organic amines) such as dicyclohexylamines, t-butyl amines, and salts with amino acids such as arginine, lysine and the like. Basic nitrogen-containing groups may be quarternized with agents such as lower alkyl halides (e.g., methyl, ethyl, and butyl chlorides, bromides and iodides), dialkyl sulfates (e.g., dimethyl, diethyl, and dibutyl sulfates), long chain halides (e.g., decyl, lauryl, and stearyl chlorides, bromides and iodides), aralkyl halides (e.g., benzyl and phenethyl bromides), and others.

The present invention further includes the compounds of Formula (I) and synthetic intermediates in all their isolated forms. For example, the above-identified compounds are intended to encompass all forms of the compounds such as, any solvates, hydrates, stereoisomers, and tautomers thereof.

As used herein, the term “composition” is intended to encompass a product comprising the specified ingredients in the specified amounts, as well as any product which results, directly or indirectly, from combination of the specified ingredients in the specified amounts.

The present invention provides processes for the preparation of the Compound of the Formula (I)), as well as synthetic intermediates which are useful in the preparation of the Compound of the Formula (I). In addition, the invention provides certain synthetic intermediates which are useful, among other things, for the preparation of the Compound of Formula (I)

In one aspect, the present invention provides processes for the preparation of the compound of Formula (I). Thus, in embodiment no. 1, the present invention provides a process comprising:

reacting amine

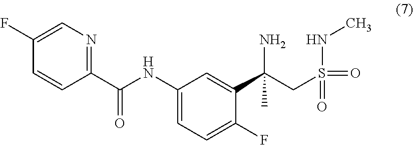 [SMILES: CNS(=O)(=O)C[C@](C)(N)c1cc(NC(=O)c2ccc(F)cn2)ccc1F]

with a cyanating agent to form the compound of Formula (I). In embodiment no. 2, the present invention provides a process as set forth in embodiment no. 1, wherein the cyanating agent is selected from the group consisting of cyanogen bromide, cyanogen fluoride, cyanogen chloride, cyanogen iodide, 2-methoxyphenyl cyanate, 4-methoxyphenyl cyanate, 4-phenylphenyl cyanate, and bisphenol A cyanate. In embodiment no. 3, the cyanating agent is cyanogen bromide.

In embodiment no. 4, the present invention provides a process as set forth in embodiment no. 1, wherein the reaction is conducted with from 1 to 2 equivalents of cyanogen bromide. In embodiment no. 5, the reaction is conducted with from 1.3 to 1.7 equivalents of cyanogen bromide.

In embodiment no. 6, the present invention provides a process as set forth in embodiment no. 1, wherein the reaction is conducted in an organic solvent selected from the group consisting of acetonitrile, acetone, toluene, dichloromethane, dichloroethane, dimethyl formamide, dimethylacetamide, dimethylsulfoxide, tetrahydrofuran, chlorobenzene, 1,2-dichlorobenzene, cyclopentylmethyl ether, ethyl acetate, isopropyl acetate, nitromethane, trifluoromethylbenzene, methyl ethyl ketone, DME, 2-methyltetrahydrofuran, pentane, N-methylpyrrolidinone, hexane, and n-heptane, or mixtures thereof. In embodiment no. 7, the organic solvent is acetonitrile. In an alternative of embodiment no. 7, the organic solvent is a mixture of acetonitrile and ethyl acetate. In another alternative of embodiment no. 7, the organic solvent is a mixture of acetonitrile and isopropyl acetate.

In embodiment no. 8, the present invention provides a process as set forth in embodiment no. 1, wherein the reaction is conducted at a temperature of 60 to 100° C. In embodiment no. 9, the reaction temperature is from 70 to 90° C.

In an alternative of embodiment no. 8 and/or 9, the reaction product includes the hydrogen bromide salt of of the compound of Formula (I), and said salt is converted to the corresponding free base and recrystallized from a suitable solvent (or combination of solvents), such as ethyl acetate and heptane. In embodiment no. 10, the present invention provides a process as set forth in embodiment no. 1, wherein the reaction product is recrystallized in a suitable solvent or combination of solvents. For example, the compound of the Formula (I) can be recrystallized from a mixture of ethyl acetate and heptane.

In embodiment no. 11, the present invention provides a process as set forth in embodiment no. 1, wherein the reaction is conducted in acetonitrile at 70-90° C. with 1 to 2 equivalents of cyanogen bromide.

In embodiment no. 12, the present invention provides a process as set forth in embodiment no. 1, wherein the amine (7) is prepared by

deprotecting a PG-protected sulfonamide

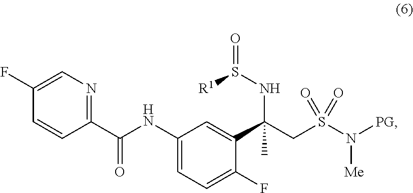

wherein

R₁ is
C₁-C₆ alkyl; or
phenyl, wherein the phenyl is unsubstituted or substituted by 1 to 3 substituents selected from the group consisting of C₁-C₄ alkyl, C₁-C₄ haloalkyl, —O—C₁-C₄ alkyl, —O—C₁-C₄ haloalkyl, halo, and nitro; and

PG is a protecting group;

with an acid to form the amine (7). In one such embodiment, the PG is butoxycarbonyl (Boc). In another such embodiment, PG is para-methoxybenzyl (PMB). In another such embodiment, the acid is methane sulfonic acid or trifluoroacetic acid. In another alternative of this embodiment, methane sulfonic acid is the acid and the protecting group is PMB.

In embodiment no. 12A, the present invention provides a process as set forth in embodiment no. 1, wherein the amine (7) is prepared by

deprotecting a PG-protected amine

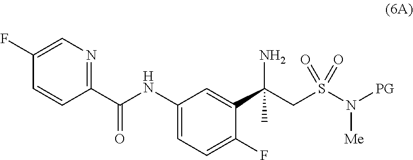

with an acid to form the amine (7), wherein PG is a protecting group. In one such embodiment, the PG is butoxycarbonyl (Boc). In another such embodiment, PG is para-methoxybenzyl (PMB). In another such embodiment, the acid is methane sulfonic acid or trifluoroacetic acid. In another alternative of this embodiment, methane sulfonic acid is the acid and the protecting group is PMB.

In embodiment no. 13, the present invention provides a process as set forth in embodiment no. 12, wherein the PG-protected sulfonamide (6), and in embodiment no. 12A, wherein the PG-protected amine (6A) is deprotected with 3 to 7 equivalents of the trifluoroacetic acid or with 3 to 7 equivalents of methanesulfonic acid.

In embodiment no. 14 the present invention provides a process as set forth in embodiment no. 12, and in embodiment no. 12A, wherein the deprotection is conducted in a solvent selected from the group consisting of acetic acid, toluene, dichloromethane, tetrahydrofuran, isopropyl acetate, dimethylacetamide, N-methylpyrrolidone, cyclopropylmethyl ether, acetonitrile, methyl tert-butyl ether, isopropanol, and mixtures thereof. In an alternative of embodiment no. 14 is embodiment no. 15, wherein the solvent is toluene. In an alternative to embodiment no. 15, the solvent is toluene and the acid is trifluoroacetic acid. In another alternative to embodiment no. 15, the solvent is acetic acid. In another alternative to embodiment no. 15, the solvent is acetic acid and the acid is methanesulfonic acid.

In embodiment no. 16 the present invention provides a process as set forth in embodiment no. 12, or in embodiment no. 12A, wherein the deprotection is conducted at 45 to 75° C., for example, at 55 to 65° C.

In embodiment no. 17 the present invention provides a process as set forth in embodiment no. 12, wherein the PG-protected sulfonamide (6), or in embodiment no. 12A, wherein the PG-protected sulfonamide (6A), is deprotected with 3 to 7 equivalents of the trifluoroacetic acid or with 3 to 7 equivalents of the methane sulfonic acid, in toluene, at 45 to 75° C., in toluene, at 45 to 75° C.

In embodiment no. 18 the present invention provides a process as set forth in embodiment no. 12, or in embodiment no. 12A, wherein the amine (7) is further purified by:

reacting the amine (7) with an enantiomerically pure chiral acid of the Formula A-H to form a diastereomeric salt mixture;

separating the salt

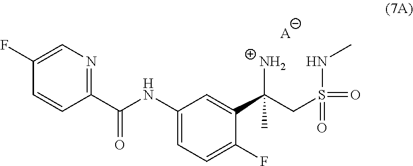

from the diastereomeric salt mixture;

reacting the salt (7A) with an aqueous base; and

recovering the free base of amine (7).

In embodiment no. 19 the present invention provides a process as set forth in embodiment no. 18, wherein the enantiomerically pure chiral acid of the Formula A-H is selected from the group consisting of L-tartaric acid, L-(+)-mandelic acid, L-(−)-malic acid, (1S)-(+)-10-camphorsulfonic acid, (−)-di-O,O-p-toluyl-L-tartaric acid, (−)-O,O-dibenzoyl-L-tartaric acid, (+)-camphoric acid, L-pyroglutamic acid, (1S)-(−)-camphanic acid, L-valine, (1S)-(+)-3-bromocamphor-10-sulfonic acid hydrate, L-histidine, D-tartaric acid, D-(−)-mandelic acid, D-(+)-malic acid, (1R)-(−)-10-camphorsulfonic acid, (+)-Di-O,O-p-toluyl-D-tartaric acid, (+)-O,O-dibenzoyl-D-tartaric acid, (−)-camphoric acid, D-pyroglutamic acid, (1R)-(+)-camphanic acid, D-valine, (+)-naproxen, and L-isoleucine.

In embodiment no. 20, the present invention provides a process as set forth in embodiment no. 15, wherein the aqueous base is sodium carbonate in water.

In embodiment no. 21, the present invention provides a process as set forth in embodiment no. 18, wherein the process comprising recrystallizing the recovered salt (7A) and isolating the recrystallized salt (7A).

In embodiment no. 22, the present invention provides a process as set forth in embodiment no. 12, wherein the PG-protected sulfonamide (6) is prepared by

coupling the aryl fluoride

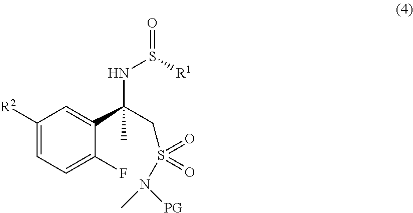

wherein
R² is halo, selected from the group consisting of bromo, chloro and iodo;
a group of the formula —O—S(O)₂—R^{2a}, wherein R^{2a} is methyl, chloromethyl, dichloromethyl, phenyl, p-trifluoromethylbenzyl, p-toluenyl, p-bromophenyl, p-fluorophenyl, p-methoxyphenyl, 2-nitrophenyl, 4-nitrophenyl, and 2,4-dichlorophenyl;
or
a diazonium group; and
PG is a protecting group;
with 5-fluoropicolinamide
   in the presence of:
   a copper or palladium reagent;
   a ligand; and

a Brønsted base to form the PG-protected sulfonamide (6). In one such embodiment, PG in compound (4) is butoxycarbonyl (Boc). In another such embodiment, PG in compound (4) is para-methoxybenzyl (PMB).

In an alternative of embodiment no. 22, the present invention provides a process as set forth in embodiment no. 12A, wherein the PG-protected amine (6A) is prepared by
coupling the aryl fluoride

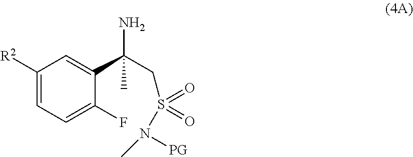

or a salt thereof;
wherein R² is:
   halo, selected from the group consisting of bromo, chloro and iodo;
   a group of the formula —O—S(O)₂—R^{2a}, wherein R^{2a} is methyl, chloromethyl, dichloromethyl, phenyl, p-trifluoromethylbenzyl, p-toluenyl, p-bromophenyl, p-fluorophenyl, p-methoxyphenyl, 2-nitrophenyl, 4-nitrophenyl, and 2,4-dichlorophenyl;
or
a diazonium group; and
PG is a protecting group;
with 5-fluoropicolinamide,
in the presence of:
   a copper or palladium reagent;
   a ligand; and

a Brønsted base to form the PG-protected amine (6A). In one such embodiment, the PG in the compound (4A) is butoxycarbonyl (Boc). In another such embodiment, PG in compound (4A) is para-methoxybenzyl (PMB). In another such embodiment, compound (4A) is in the form of an acid addition salt and PG is Boc or PMB, wherein said acid addition salt is selected from those defined hereinabove. In another embodiment, the aryl fluoride (4A) is in the form of a (−)-O,O-dibenzoyl-L-tartrate salt. In another embodiment, the aryl fluoride (4A) is in the form of a (−)-O,O-dibenzoyl-L-tartrate salt and PG is PMB. In embodiment no. 23, the present invention provides a process as set forth in embodiment no. 22 or the alternative embodiment thereof, wherein the coupling is conducted with 1 to 3 equivalents of 5-fluoropicolinamide.

In embodiment no. 24, the present invention provides a process as set forth in embodiment no. 22 or the alternative embodiment thereof, wherein the copper or palladium reagent is selected from the group consisting of CuI, CuI-TBAI, CuBr, CuPF₆(MeCN)₄, CuBr₂, [Cu(OTf)]₂-tol, CuCl, Cu metal, Cu₂O, Cu(OAc)₂, (aminobiphenyl)PdOMs dimer, and (aminobiphenyl)PdCl dimer.

In embodiment no. 25, the present invention provides a process as set forth in embodiment no. 22 or the alternative embodiment thereof, wherein the copper or palladium reagent is CuI. In one such embodiment, said CuI is present at in least 0.01 equivalents. In another such embodiment, said CuI is present in at least 0.2 equivalents. In a non-limiting example, said CuI is present in from 0.01 to 1.4 equivalents. In another example, said CuI is present in from 0.2 to 1.4 equivalents.

In embodiment no. 26, the present invention provides a process as set forth in embodiment no. 22 or the alternative embodiment thereof, wherein the ligand is selected from the group consist of N,N′-dimethyl diaminocyclohexane, N,N′-dimethylethylenediamine, diaminocyclohexane, tBuBrettphos, DMEDA, Xphos, RuPhos, Sphos, water-soluble Sphos, tBuXPhos, Rockphos, Brettphos, AdBrettphos, Qphos, MorDalphos, Amphos, CataCXiumA, tBu₃P, Cy₃P, MeCgPPh, o-tol₃P, PPh₃, BINAP, dppf, dtbpf, Josiphos SL-J009, Johnphos, Xantphos, and NiXantphos. In embodiment no. 27, the ligand is N,N′-dimethyldiaminocyclohexane or N,N′-dimethylethylenediamine.

In embodiment no. 28, the present invention provides a process as set forth in embodiment no. 22 or the alternative embodiment thereof, wherein the Brønsted base is selected from the group consisting of potassium carbonate, potassium phosphate, cesium carbonate, and potassium bicarbonate. In embodiment no. 29, the Brønsted base is potassium carbonate.

In embodiment no. 30, the present invention provides a process as set forth in embodiment no. 22 or the alternative embodiment thereof, wherein the coupling is conducted in a high boiling organic solvent. For example, the coupling is conducted in a solvent selected from the group consisting of toluene, dimethylacetamide, t-amyl alcohol, and cyclopentyl methyl ether. In embodiment no. 31, the high boiling solvent is toluene.

In embodiment no. 32, the present invention provides a process as set forth in embodiment no. 22 or the alternative embodiment thereof, wherein the coupling is conducted at 70 to 130° C., for instance, at 80 to 110° C.

In embodiment no. 33, the present invention provides a process as set forth in embodiment no. 22 or the alternative embodiment thereof, wherein the coupling is optionally conducted in the presence of an additive selected from the group consisting of NaI, KI, I₂ and TBAI.

In embodiment no. 34, the present invention provides a process as set forth in embodiment no. 22 or the alternative embodiment thereof, wherein the copper or palladium reagent is CuI present at 0.01 to 1.4 equivalents, the ligand is N,N′-dimethyldiaminocyclohexane, and the Brønsted base is potassium carbonate. In embodiment no. 35, the a process as set forth in embodiment no. 34, the coupling is conducted in toluene at 70 to 130° C.

In embodiment no. 36, the present invention provides a process as set forth in embodiment no. 22 or the alternative embodiment thereof, wherein the amine (7) is further purified by separating the R-enantiomer from the mixture of enantiomers. In embodiment no. 37, the separation of the R-enantiomer of the amine (7) from the mixture comprises recrystallization. Suitable recrystallization solvents for this separation include, for example, a mixture of acetonitrile/methyl tert-butyl ether, and toluene (neat). The separation of the R-enantiomer of the amine (7) from the mixture can also be performed using chromatography on a chiral solid-phase media.

In embodiment no. 38, the present invention provides a process as set forth in embodiment no. 22 or the alternative embodiment thereof, wherein the aryl fluoride (4) or aryl fluoride (4A) is prepared by:

treating the methylsulfonamide

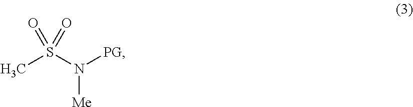

wherein PG is a protecting group, with an alkali metal base to form the alkali metalate species of the methylsulfonamide (3); and

reacting the alkali metalate species of the methylsulfonamide (3) with sulfinyl imine

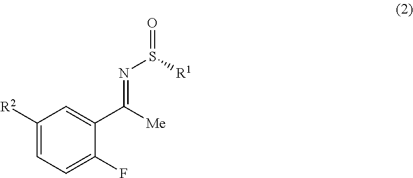

to form the aryl fluoride (4). In one such embodiment, PG is Boc. In another such embodiment, PG is PMB. In the aforementioned alternative to embodiment no. 38, the aryl fluoride (4) is further treated with a strong acid to form (4A). Suitable strong acids will be readily apparent to those of ordinary skill in the art. Non-limiting examples of such acids include, but are not limited to, hydrochloric acid, hydrobromic acid, methanesulfonic acid, tetrafluoroboric acid, trifluoromethane sulfonic acid, sulfuric acid, fumaric acid, and citric acid. In one such embodiment, the strong acid is hydrochloric acid. In embodiment no. 38, the aryl fluoride (4) is produced according to the aforementioned process in the absence of treating with an acid. In another alternative of each of the immediately preceeding embodiments, PG is PMB.

In embodiment no. 39, the present invention provides a process as set forth in embodiment no. 38, wherein the alkali metal base is selected from the group consisting of n-HexLi, n-BuLi, KHMDS, and NaHMDS. In embodiment no. 40, the alkali metal base is n-BuLi. In an alternative to embodiment no. 40, the alkali metal base is n-hexLi.

In embodiment no. 41, the present invention provides a process as set forth in embodiment no. 38, wherein the treatment with the alkali metal base and reaction with the sulfinyl imine (2) is conducted in an organic solvent selected from the group consisting of dichloromethane, tetrahydrofuran, 2-methyltetrahydrofuran, toluene, cyclopentylmethyl ether, and dimethoxyethane. In embodiment no. 42, the organic solvent is tetrahydrofuran.

In embodiment no. 43, the present invention provides a process as set forth in embodiment no. 38, wherein the treatment with the alkali metal base and reaction with the sulfinyl imine (2) is optionally conducted in the presence of an additive selected from the group consisting of MgCl₂, ZnCl₂, Al(O-iPr)₃, In(OTf)₃, FeCl₂, CuBr, CuBr₂, SnCl₂, Sc(OTf)₃, Fe(acac)₃, BF₃.OEt₂, Ti(OEt)₄, TMSOTf, TMEDA, HMPA and LiCl.

In embodiment no. 44, the the present invention provides a process as set forth in embodiment no. 38, wherein the alkali metal base is n-BuLi and the treatment with the alkali metal base and reaction with the sulfinyl imine (2) is conducted in THF. In an alternate of embodiment no. 44, the alkali metal base is n-HexLi and the treatment with the alkali metal base and reaction with the sulfinyl imine (2) is conducted in THF.

In embodiment no. 45, the present invention provides a process as set forth in embodiment no. 38, wherein the sulfinyl imine (2) is prepared by:

condensing ketone

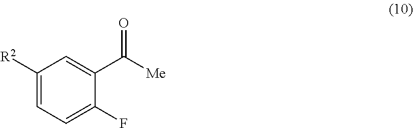

with

a sulfinamide of the formula R¹—S(O)—NH₂ (11) in the presence of a tetra(C₁-C₆ alkoxy)titanium (IV) or tetra(C₁-C₆ alkoxy)zirconium (IV) catalyst to form sulfinyl imine (2).

In embodiment no. 46, the present invention provides a process as set forth in embodiment no. 45, wherein the condensation catalyst is titanium (IV) ethoxide.

In embodiment no. 47, the present invention provides a process as set forth in embodiment no. 45, wherein the condensation is conducted in a solvent selected from the group consisting of ethyl acetate, acetone, toluene, dichloromethane, dichloroethane, dimethylformamide, dimethylacetamide, dimethylsulfoxide, tetrahydrofuran, chlorobenzene, 1,2-dichlorobenzene, cyclopentylmethyl ether, acetonitrile, isopropyl acetate, nitromethane, trifluoromethyl benzene, methyl ethyl ketone, dimethoxyethane, 2-methyltetrahydrofuran, pentane, N-methylpyrrolidone, hexane and n-heptane. In embodiment no. 48, the solvent for the condensation is ethyl acetate.

In embodiment no. 49, the present invention provides a process as set forth in embodiment no. 45, wherein the catalyst is titanium (IV) ethoxide and the condensation is conducted in ethyl acetate. In embodiment no. 50, the process as set forth in embodiment no. 49, wherein the condensation is conducted at 40 to 70° C.

In embodiment no. 51, the present invention provides a process as set forth in embodiment no. 12 or in embodiment no. 12A, wherein the PG-protected sulfonamide (6) or the PG-protected amine (6A) is prepared by:

treating the methylsulfonamide

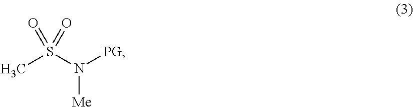

wherein PG is a protecting group, with an alkali metal reagent to form the alkali metalated species of the methylsulfonamide (3); and

reacting the alkali metalated species of the methylsulfonamide (3) with sulfinyl imine

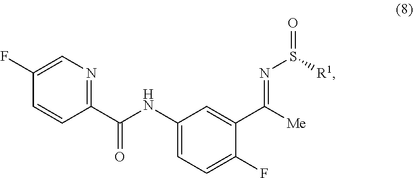

wherein
R₁ is
   C₁-C₆ alkyl; or
   phenyl, wherein the phenyl is unsubstituted or substituted by 1 to 3 substituents selected from the group consisting of C₁-C₄ alkyl, C₁-C₄ haloalkyl, —O—C₁-C₄ alkyl, —O—C₁-C₄ haloalkyl, halo, and nitro;

to form the PG-protected sulfonamide (6), which can be further treated with a strong acid to form the PG-protected amine (6A). Suitable strong acids will be readily apparent to those of ordinary skill in the art. Non-limiting examples of such acids include, but are not limited to, hydrochloric acid, hydrobromic acid (HBr), methanesulfonic acid, tetrafluoroboric acid, trifluoromethane sulfonic acid, sulfuric acid, fumaric acid, and citric acid. In one such embodiment, the strong acid is hydrochloric acid (HCl). In embodiment no. 51, PG-protected sulfonamide (6) is produced according to the aforementioned process in the absence of treating with an acid. In an alternative of each of the immediately preceeding embodiments, PG is PMB.

In embodiment no. 52, the present invention provides a process as set forth in embodiment no. 51, wherein the alkali metal base is selected from the group consisting of n-HexLi, n-BuLi, KHMDS, and NaHMDS. In embodiment no. 53, the alkali metal base is n-BuLi. In an alternative to embodiment no. 52, the alkali metal base is n-hexLi.

In embodiment no. 54, the present invention provides a process as set forth in embodiment no. 51, wherein the treatment with the alkali metal base and reaction with the sulfinyl imine (8) is conducted in an organic solvent selected from the group consisting of dichloromethane, tetrahydrofuran, 2-methyltetrahydrofuran, toluene, cyclopentylmethyl ether, and dimethoxyethane. In embodiment no. 55, the organic solvent is dichloromethane.

In embodiment no. 56, the present invention provides a process as set forth in embodiment no. 51, wherein the treatment with the alkali metal base and reaction with the sulfinyl imine (8) is optionally conducted in the presence of an additive selected from the group consisting of MgCl₂, ZnCl₂, Al(O-iPr)₃, In(OTf)₃, FeCl₂, CuBr, CuBr₂, SnCl₂, Sc(OTf)₃, Fe(acac)₃, BF₃.OEt₂, Ti(OEt)₄, TMSOTf, TMEDA, HMPA and LiCl.

In embodiment no. 57, the present invention provides a process as set forth in embodiment no. 51, wherein the alkali metal base is n-BuLi and the treatment with n-BuLi and reaction with the sulfinyl imine (8) is conducted in dichloromethane. In an alternative to embodiment no. 57, the alkali metal base is n-HexLi.

In embodiment no. 58, the present invention provides a process as set forth in embodiment no. 51, wherein sulfinyl imine (8) is prepared by condensing ketone

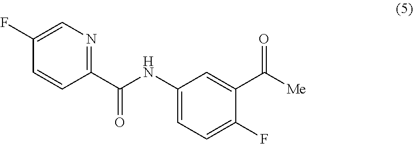 [SMILES: CC(=O)c1cc(NC(=O)c2ccc(F)cn2)ccc1F]

with a sulfinamide of the formula R¹—S(O)—NH₂ (11) in the presence of a tetra(C₁-C₆ alkoxy)titanium (IV) catalyst to form sulfinyl imine (8).

In embodiment no. 59, the present invention provides a process as set forth in embodiment no. 58, wherein the catalyst is Ti(OEt)₄ and the condensation is conducted in tetrahydrofuran.

In embodiment no. 60, the present invention provides a process as set forth in embodiment no. 58, wherein ketone (5) is prepared by coupling ketone

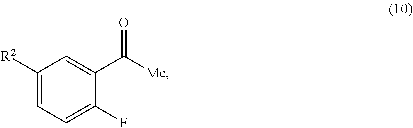

wherein R² is:
(i) halo, selected from the group consisting of bromo, chloro and iodo;
(ii) a group of the formula —O—S(O)₂—R^{2a}, wherein R^{2a} is methyl, chloromethyl, dichloromethyl, phenyl, p-trifluoromethylbenzyl, p-toluenyl, p-bromophenyl, p-fluorophenyl, p-methoxyphenyl, 2-nitrophenyl, 4-nitrophenyl, and 2,4-dichlorophenyl; or
(iii) a diazonium group;
   with 5-fluoropicolinamide, in the presence of:
a copper reagent;
a ligand; and
a Brønsted base;
   to form the ketone (5).

In embodiment no. 61, the present invention provides a process as set forth in embodiment no. 60, wherein the copper reagent is selected from the group consisting of CuI, CuI-TBAI, Cu(TMHD)₂, Cu(AcChxn)₂, and Cu(iBuChxn)₂. In embodiment no. 62, the copper reagent is CuI.

In embodiment no. 63, the present invention provides a process as set forth in embodiment no. 60, wherein the ligand is selected from the group consisting of DMEDA, DACH, DM-DACH, N,N-dimethylglycine, TMEDA, bipyridine, 4,4-di-tBubipy, phenanthroline, neocuprine, tetramethylphenanthroline, terpyridine, tri-T-tBu-typyridine, 8-hydroxyquinoline, proline, picolinic acid, thiophene-2-carboxylic acid, N,N-diethylsalicylamide, SALOX, Chxn-Py-Al, 1,3-di(2-pyridyl)-1,3-propanedione, TMHD, AcChxn, and iBuChxn. In embodiment no. 64, the ligand is selected from the group consisting of DMEDA, DACH, DM-DACH, and N,N-dimethylglycine.

In embodiment no. 65, the present invention provides a process as set forth in embodiment no. 60, wherein the Brønsted base is selected from the group consisting of potassium carbonate, cesium carbonate, and potassium phosphate.

In embodiment no. 66, the present invention provides a process as set forth in embodiment no. 60, wherein the coupling is conducted in a solvent selected from the group consisting of toluene and DMSO.

In embodiment no. 67, the present invention provides a process as set forth in embodiment no. 60, wherein the copper reagent is as set forth in embodiment no. 61, the ligand is as set forth in embodiment no. 64, the Brønsted base is as set forth in embodiment no. 65, and the coupling is conducted in the solvents set forth in embodiment no. 66.

In embodiment no. 68, the present invention provides a process as set forth in embodiment no. 60, wherein the ketone (5) is prepared by coupling 1-(5-amino-2-fluorophenyl)ethanone with 5-fluoropicolinic acid.

In embodiment no. 69, the present invention provides a process as set forth in embodiment no. 68, wherein the the coupling is conducted with a coupling agent selected from propylphosphonic anhydride (T₃P), DCC and EDC.

In embodiment no. 70, the present invention provides a process as set forth in embodiment no. 1, wherein the amine (7) is prepared by coupling the amine

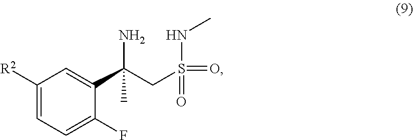

wherein R² is:
(i) halo, selected from the group consisting of bromo, chloro and iodo;
(ii) a group of the formula —O—S(O)₂—R^{2a}, wherein R^{2a} is methyl, chloromethyl, dichloromethyl, phenyl, p-trifluoromethylbenzyl, p-toluenyl, p-bromophenyl, p-fluorophenyl, p-methoxyphenyl, 2-nitrophenyl, 4-nitrophenyl, and 2,4-dichlorophenyl; or
(iii) a diazonium group;

with 5-fluoropicolinamide in the presence of:

a metal reagent selected from the group consisting of a palladium reagent and a copper reagent
a ligand; and
a Brønsted base.

In embodiment no. 71, the present invention provides a process as set forth in embodiment no. 70, wherein the metal reagent is selected from the group consisting of CuI-TBAI, CuBr, CuPF₆(CH₃CN)₄, CuBr₂, [Cu(OTf))]₂-tol, CuCl, CuI, CuBr-DMS, (aminobiphenyl)PdOMs dimer, and (aminobiphenyl)PdCl dimer. In embodiment no. 72, the metal reagent is a palladium reagent selected from the group consisting of (aminobiphenyl)PdOMs dimer, and (aminobiphenyl)PdCl dimer.

In embodiment no. 73, the present invention provides a process as set forth in embodiment no. 70, wherein the ligand is selected from the group consisting of N,N′-dimethyl diaminocyclohexane, diaminocyclohexane, DMEDA, Rockphos, tBuBrettphos, AdBrettphos, Xphos, RuPhos, Sphos, water-soluble Sphos, tBuXPhos, Brettphos, Qphos, MorDalphos, Amphos, CataCXiumA, tBu₃P, Cy₃P, MeCgPPh, o-tol₃P, PPh₃, BINAP, dppf, dtbpf, Josiphos SL-J009, Johnphos, Xantphos, and NiXantphos.

In embodiment no. 74, the present invention provides a process as set forth in embodiment no. 70, wherein the coupling is conducted in a solvent selected from the group consisting of 2-methyltetrahydrofuran, toluene, dimethylacetamide, t-amyl alcohol, and cyclopentyl methyl ether.

In embodiment no. 75, the present invention provides a process as set forth in embodiment no. 70, wherein the amine (9) is prepared by deprotecting the aryl fluoride (4) or by deprotecting the aryl fluoride (4A) with trifluoroacetic acid to form the amine (9). For example, in embodiment no. 76, the aryl fluoride (4) or the aryl fluoride (4A) is reacted with 3 to 7 equivalents of the trifluoroacetic acid.

In embodiment no. 77, the present invention provides a process as set forth in embodiment no. 75, wherein the deprotection is conducted in a solvent selected from the group consisting of toluene toluene, dichloromethane, tetrahydrofuran, isopropyl acetate, dimethylacetamide, N-methylpyrrolidone, cyclopropylmethyl ether, acetonitrile, methyl ten-butyl ether, and isopropanol.

In embodiment no. 78, the present invention provides a process as set forth in embodiment no. 73, wherein the deprotection is conducted at 45 to 75° C., for instance, at 55 to 65° C.

In embodiment no. 79, the present invention provides a process as set forth in embodiment no. 75, wherein the aryl fluoride (4) or the aryl fluoride (4A) is reacted with 3 to 7 equivalents of the trifluoroacetic acid, the deprotection is conducted in a solvent as set forth in embodiment no. 77; and at temperature as set forth in embodiment no. 78.

In embodiment no. 80, the present invention provides a process as set forth in any one of embodiment nos. 22, 38, 45, 51, and 58, wherein R¹ is tert-butyl.

In embodiment no. 81, the present invention provides a process as set forth in any one of embodiment nos. 22, 38, 45, 60, and 70, wherein R² is bromo.

In embodiment no. 82, the present invention provides a process as set forth in any one of embodiment nos. 22, 38, and 45, wherein R¹ is tert-butyl and R² is bromo.

In embodiment no. 83, the present invention provides a process for preparing the compound of Formula (I), wherein:

the amine (7) is reacted with the cyanating agent as set forth in embodiment no. 11;

the amine (7) is prepared by deprotecting PG-protected sulfonamide (6) or PG-protected amine (6A) as set forth in any one of embodiment nos. 12-21; and

R¹ is tert-butyl. In another such embodiment, PG is PMB.

In embodiment no. 84, the present invention provides a process for preparing the compound of Formula (I) wherein:

the amine (7) is reacted with the cyanating agent as set forth in embodiment no. 11;

the amine (7) is prepared by deprotecting PG-protected sulfonamide (6) or by deprotecting PG-protected amine (6A) as set forth in embodiment no. 17;

the PG-protected sulfonamide (6) or the PG-protected amine (6A) is prepared by coupling the aryl fluoride (4) or the aryl fluoride (4A) with 5-fluoropicolinamide as set forth in any one of embodiment nos. 22-37; and

R¹ is tert-butyl and R² is bromo. In another such embodiment, PG is PMB.

In embodiment no. 85, the present invention provides a process for preparing the compound of Formula (I) wherein:

the amine (7) is reacted with the cyanating agent as set forth in embodiment no. 11;

the amine (7) is prepared by deprotecting the PG-protected sulfonamide (6) or by deprotecting the PG-protected amine (6A) as set forth in embodiment no. 17;

the PG-protected sulfonamide (6) is prepared by coupling the aryl fluoride (4) with 5-fluoropicolinamide as set forth embodiment no. 34 (or the PG-protected amine (6A) is prepared by coupling the aryl fluoride (4A) as set forth in embodiment no. 34); and

R¹ is tert-butyl and R² is bromo. In another such embodiment, PG is PMB.

In another aspect, the present invention provides processes for the preparation of certain synthetic intermediates useful in the preparation of the compound of Formula (I). Thus, in embodiment no. 86, the present invention provides a process for preparing the amine (7) from PG-protected sulfonamide (6) or from PG-protected amine (6A), under the conditions set forth in any one of embodiment nos. 12-21. In embodiment no. 87, the present invention provides the process for preparing amine (7) as set forth in embodiment no. 86, wherein PG-protected sulfonamide (6) is prepared from aryl fluoride (4) and 5-fluoropicolinamide as set forth in any one of embodiment nos. 22-37. In an alternative embodiment no. 87, the present invention provides the process for preparing amine (7) as set forth in embodiment no. 86, wherein PG-protected amine (6A) is prepared from aryl fluoride (4A) and 5-fluoropicolinamide as set forth in any one of embodiment nos. 22-37. In embodiment no. 88, the present invention provides a process for preparing the amine (7) as set forth in embodiment no. 86, wherein R¹ is tert-butyl. In embodiment no. 89, the present invention provides a process for preparing the amine (7) as set forth in embodiment no. 87, wherein R¹ is tert-butyl and R² is bromo. In another alternative of each of the immediately preceeding embodiments, PG is PMB.

In embodiment no. 90, the present invention provides a process for preparing the PG-protected sulfonamide (6) from the aryl fluoride (4) under the conditions set forth in any one of embodiment nos. 22-37. In an alternative of embodiment no. 90, the present invention provides a process for preparing the PG-protected amine (6A) from the aryl fluoride (4A) under the conditions set forth in any one of embodiment nos. 22-37. In embodiment no. 91, the present invention provides a process for preparing the PG-protected sulfonamide (6) as set forth in embodiment no. 90, wherein R¹ is tert-butyl and R² is bromo. In an alternative of embodiment no. 91, the present invention provides a process for preparing the PG-protected amine (6A) as set forth in alternative embodiment no. 90, wherein R² is bromo. In another alternative of each of the immediately preceeding embodiments, PG is PMB.

In another aspect, the present invention provides synthetic intermediates useful in the preparation of the compound of Formula (I). Thus, in embodiment no. 92, the invention provides amine (7) or a salt thereof.

In another embodiment, the present invention provides amine

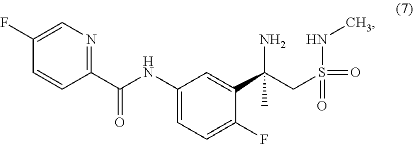 [SMILES: CNS(=O)(=O)C[C@](C)(N)c1cc(NC(=O)c2ccc(F)cn2)ccc1F]

or a salt thereof. In one embodiment, the salt is an acid addition salt of amine (7). In one embodiment, said salt is selected from acetate, ascorbate, benzoate, benzenesulfonate, bisulfate, borate, butyrate, citrate, camphorate, camphorsulfonate, fumarate, hydrochloride, hydrobromide, hydroiodide, lactate, maleate, methanesulfonate, naphthalenesulfonate, nitrate, oxalate, phosphate, propionate, salicylate, succinate, sulfate, tartrate, thiocyanate, toluenesulfonate (also known as tosylate), and 1-hydroxy-2-naphthoate.

In another embodiment, the present invention provides PG-protected sulfonamide

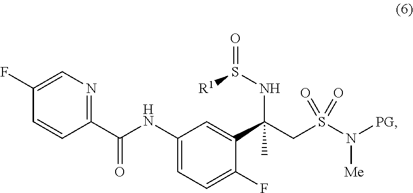

or a salt thereof, wherein PG is a protecting group; and R₁ is C₁-C₆ alkyl; or phenyl, wherein the phenyl is unsubstituted or substituted by 1 to 3 substituents selected from the group consisting of C₁-C₄ alkyl, C₁-C₄ haloalkyl, —O—C₁-C₄ alkyl, —O—C₁-C₄ haloalkyl, halo, and nitro. In another such embodiment, R¹ is tert-butyl. In one embodiment, the salt is an acid addition salt of PG-protected sulfonamide (6). In one embodiment, said salt is selected from acetate, ascorbate, benzoate, benzenesulfonate, bisulfate, borate, butyrate, citrate, camphorate, camphorsulfonate, fumarate, hydrochloride, hydrobromide, hydroiodide, lactate, maleate, methanesulfonate, naphthalenesulfonate, nitrate, oxalate, phosphate, propionate, salicylate, succinate, sulfate, tartrate, thiocyanate, toluenesulfonate (also known as tosylate), and 1-hydroxy-2-naphthoate. In another such embodiment, PG is selected from the group consisting of —S(O)₂R⁸, —C(O)OR⁸, —C(O)R⁸, —CH₂OCH₂CH₂SiR⁸, and —CH₂R₈ where R⁸ is selected from the group consisting of —C₁₋₈ alkyl (straight or branched), —C₃₋₈ cycloalkyl, —CH₂(aryl), and —CH(aryl)₂, wherein each aryl is independently phenyl or naphthyl and each said aryl is optionally independently unsubstituted or substituted with one or more (e.g., 1, 2, or 3) groups independently selected from —OMe, Cl, Br, and I. In another such embodiment, PG is selected from the group consisting of butoxycarbonyl (Boc) and para-methoxybenzyl (PMB). In another such embodiment, PG is para-methoxybenzyl (PMB).

In another embodiment, the present invention provides PG-protected amine

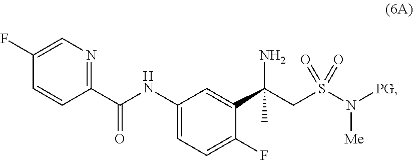

or a salt thereof. In one embodiment, the salt is an acid addition salt of PG-protected amine (6A). In one embodiment, said salt is selected from acetate, ascorbate, benzoate, benzenesulfonate, bisulfate, borate, butyrate, citrate, camphorate, camphorsulfonate, fumarate, hydrochloride, hydrobromide, hydroiodide, lactate, maleate, methanesulfonate, naphthalenesulfonate, nitrate, oxalate, phosphate, propionate, salicylate, succinate, sulfate, tartrate, thiocyanate, toluenesulfonate (also known as tosylate), and 1-hydroxy-2-naphthoate. In another such embodiment is a compound (6A) or a salt thereof and PG is selected from the group consisting of —S(O)₂R⁸, —C(O)OR⁸, —C(O)R⁸, —CH₂OCH₂CH₂SiR⁸, and —CH₂R₈ where R⁸ is selected from the group consisting of —C₁₋₈ alkyl (straight or branched), —C₃₋₈ cycloalkyl, —CH₂(aryl), and —CH(aryl)₂, wherein each aryl is independently phenyl or naphthyl and each said aryl is optionally independently unsubstituted or substituted with one or more (e.g., 1, 2, or 3) groups independently selected from —OMe, Cl, Br, and I. In another such embodiment, PG is selected from the group consisting of butoxycarbonyl (Boc) and para-methoxybenzyl (PMB). In another such embodiment, PG is para-methoxybenzyl (PMB).

In another embodiment, the present invention provides the salt

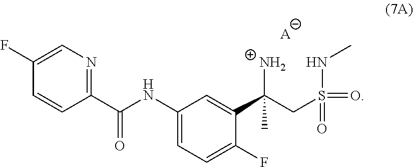

Specific non-limiting examples of such salts include the salts of any of the following acids: L-tartaric acid, L-(+)-mandelic acid, L-(−)-malic acid, (1S)-(+)-10-camphorsulfonic acid, (−)-di-O,O-p-toluyl-L-tartaric acid, (−)-O,O-dibenzoyl-L-tartaric acid, (+)-camphoric acid, L-pyroglutamic acid, (1S)-(−)-camphanic acid, L-valine, (1S)-(+)-3-bromocamphor-10-sulfonic acid hydrate, L-histidine, D-tartaric acid, D-(−)-mandelic acid, D-(+)-malic acid, (1R)-(−)-10-camphorsulfonic acid, (+)-Di-O,O-p-toluyl-D-tartaric acid, (+)-O,O-dibenzoyl-D-tartaric acid, (−)-camphoric acid, D-pyroglutamic acid, (1R)-(+)-camphanic acid, D-valine, (+)-naproxen, and L-isoleucine.

In another embodiment, the present invention provides aryl fluoride

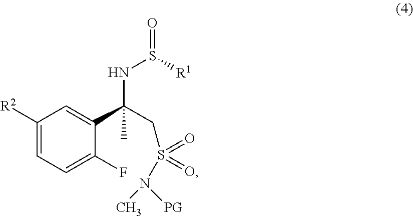

wherein

R₁ is:
C₁-C₆ alkyl; or
phenyl, wherein the phenyl is unsubstituted or substituted by 1 to 3 substituents selected from the group consisting of C₁-C₄ alkyl, C₁-C₄ haloalkyl, —O—C₁-C₄ alkyl, —O—C₁-C₄ haloalkyl, halo, and nitro;

R² is:
(i) halo, selected from the group consisting of bromo, chloro and iodo;
(ii) a group of the formula —O—S(O)₂—R^{2a}, wherein R^{2a} is methyl, chloromethyl, dichloromethyl, phenyl, p-trifluoromethylbenzyl, p-toluenyl, p-bromophenyl, p-fluorophenyl, p-methoxyphenyl, 2-nitrophenyl, 4-nitrophenyl, and 2,4-dichlorophenyl; or

a diazonium group; and

PG is selected from the group consisting of —S(O)₂R⁸, —C(O)OR⁸, —C(O)R⁸, —CH₂OCH₂CH₂SiR⁸, and —CH₂R₈ where R⁸ is selected from the group consisting of —C₁₋₈ alkyl (straight or branched), —C₃₋₈ cycloalkyl, —CH₂(aryl), and —CH(aryl)₂, wherein each aryl is independently phenyl or naphthyl and each said aryl is optionally independently unsubstituted or substituted with one or more (e.g., 1, 2, or 3) groups independently selected from —OMe, Cl, Br, and I. In another such embodiment, PG is selected from the group consisting of butoxycarbonyl (Boc) and para-methoxybenzyl (PMB). In another such embodiment of the compound (4), PG is para-methoxybenzyl (PMB).

In another embodiment, the present invention provides aryl fluoride

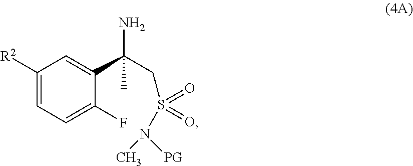

or a salt thereof, wherein

R² is:
(i) halo, selected from the group consisting of bromo, chloro and iodo;
(ii) a group of the formula —O—S(O)₂—R^{2a}, wherein R^{2a} is methyl, chloromethyl, dichloromethyl, phenyl, p-trifluoromethylbenzyl, p-toluenyl, p-bromophenyl, p-fluorophenyl, p-methoxyphenyl, 2-nitrophenyl, 4-nitrophenyl, and 2,4-dichlorophenyl; or

a diazonium group; and

and

PG is selected from the group consisting of —S(O)₂R⁸, —C(O)OR⁸, —C(O)R⁸, —CH₂OCH₂CH₂SiR⁸, and —CH₂R₈ where R⁸ is selected from the group consisting of —C₁₋₈ alkyl (straight or branched), —C₃₋₈ cycloalkyl, —CH₂(aryl), and —CH(aryl)₂, wherein each aryl is independently phenyl or naphthyl and each said aryl is optionally independently unsubstituted or substituted with one or more (e.g., 1, 2, or 3) groups independently selected from —OMe, Cl, Br, and I. In one embodiment, the compound (4A) is in the form of an acid addition salt. In another embodiment, the salt of aryl fluoride (4A) is in the form of the (−)-O,O-dibenzoyl-L-tartrate salt. In another such embodiment of the compound (4A) or a salt thereof, PG is selected from the group consisting of butoxycarbonyl (Boc) and para-methoxybenzyl (PMB). In another such embodiment of the compound (4A), PG is para-methoxybenzyl (PMB).

In another embodiment, the present invention provides sulfinyl imine

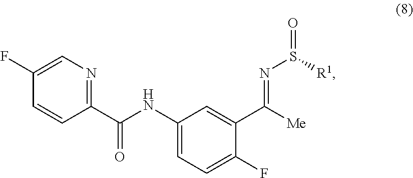

wherein

R₁ is C₁-C₆ alkyl; or phenyl, wherein the phenyl is unsubstituted or substituted by 1 to 3 substituents independently selected from the group consisting of C₁-C₄ alkyl, C₁-C₄ haloalkyl, —O—C₁-C₄ alkyl, —O—C₁-C₄ haloalkyl, halo, and nitro. In one such embodiment, R¹ is t-butyl.

In another embodiment, the present invention provides ketone

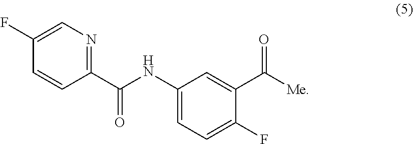 [SMILES: CC(=O)c1cc(NC(=O)c2ccc(F)cn2)ccc1F]

WO2011044181 discloses a compound of Formula (9) wherein R² is Br. In another embodiment, the present invention provides amine

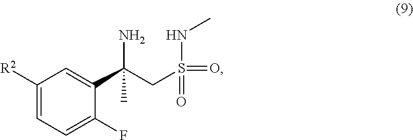

wherein R² is:
(iv) a group of the formula —O—S(O)₂—R^{2a}, wherein R^{2a} is methyl, chloromethyl, dichloromethyl, phenyl, p-trifluoromethylbenzyl, p-toluenyl, p-bromophenyl, p-fluorophenyl, p-methoxyphenyl, 2-nitrophenyl, 4-nitrophenyl, and 2,4-dichlorophenyl; or
(v) a diazonium group.

The compound of Formula (I) which is prepared by the processes of the present invention may be incorporated into suitable pharmaceutical dosage forms. Descriptions of suitable dosage forms are set forth in U.S. Pat. No. 8,729,071 at column 53, line 58 to column 55, line 14.

The following schemes further illustrate the processes of the invention. The variables R¹ and R² in the schemes are as set forth above in embodiment nos. 1-96. In the schemes and preparative examples that follow, the protecting group (where present) is depicted as PMB (para-methoxybenzyl). However, it shall be understood that, in each occurrence, PMB may be substituted with Boc or any other suitable protecting group PG, examples of which will be readily apparant to those of ordinary skill in the art. Additional non-limiting examples of suitable protecting groups include —S(O)₂R⁸, —C(O)OR⁸, —C(O)R⁸, —CH₂OCH₂CH₂SiR⁸, and —CH₂R₈ where R⁸ is selected from the group consisting of —C₁₋₈ alkyl (straight or branched), —C₃₋₈ cycloalkyl, —CH₂(aryl), and —CH(aryl)₂, wherein each aryl is independently phenyl or naphthyl and optionally substituted with one or more (e.g., 1, 2, or 3) groups independently selected from —OMe, Cl, Br, and I, as described hereinabove.

Scheme 1 shows one embodiment of the invention wherein the compound of the Formula (I) is prepared.

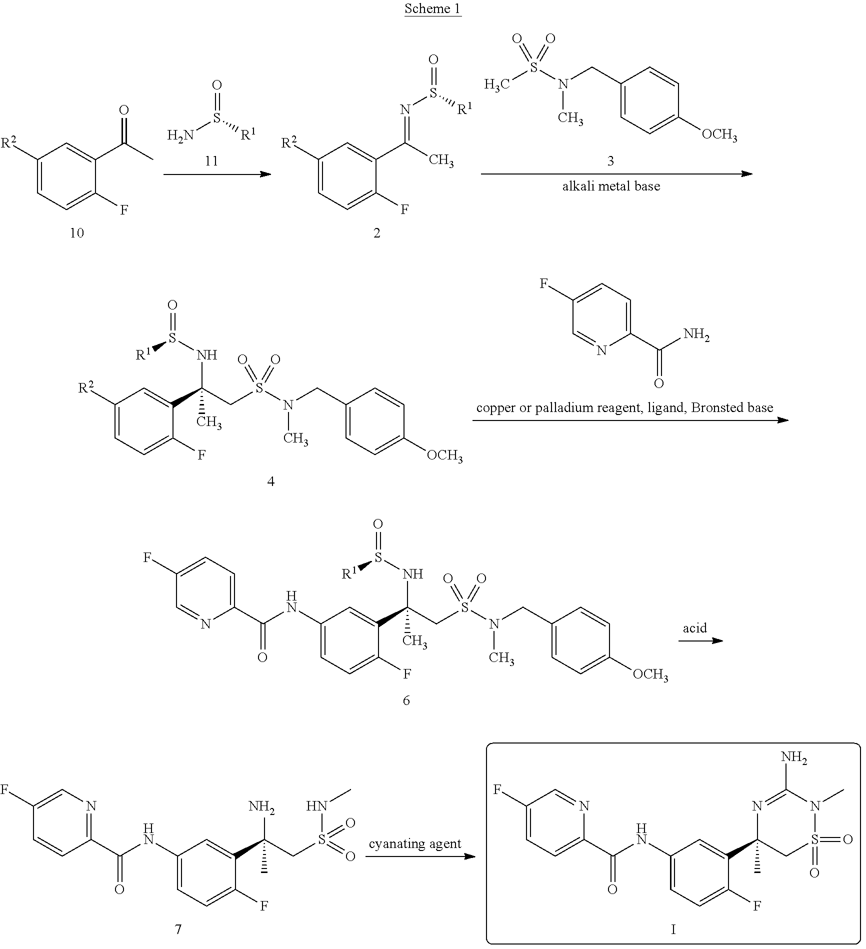

As shown in Scheme 1, substituted ketone (10) is condensed with a sulfonamide (11) to form sulfinyl imine (2). The sulfinyl imine (2) is reacted with an alkali-metalated species of the methyl sulfonamide (3) to form the aryl fluoride (4). In order to prepare the PMB-protected sulfonamide (6), the aryl fluoride (4) is coupled with a copper or palladium reagent, a ligand, and a Brønsted base. The PMB-protected sulfonamide (6) is deprotected with methanesulfonic acid to provide the amine (7). Ring cyclization is accomplished by treatment of amine (7) with a cyanating agent.

Scheme 2 illustrates one embodiment of an alternative process for preparing the amine (7).

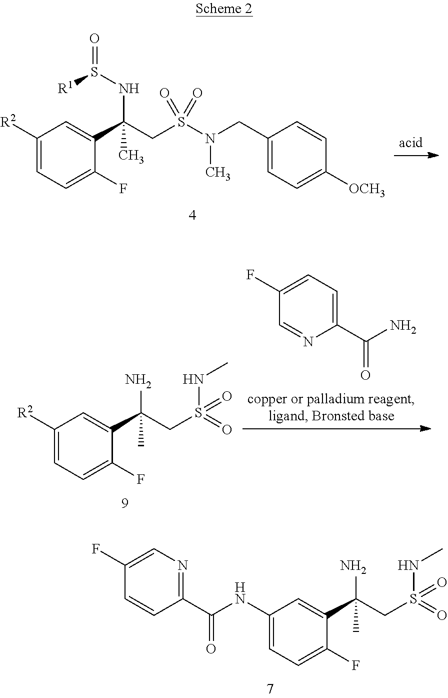

As shown in Scheme 2, the aryl fluoride (4) is deprotected with an acid such as methanesulfonic acid to provide the amine (9). Coupling of (9) with 5-fluoropicolinamide in the presence of a copper or palladium reagent, a ligand and a Brønsted base provides the amine (7).

Scheme 3 illustrates one embodiment of an alternative process for preparing the amine (6) or (6A).

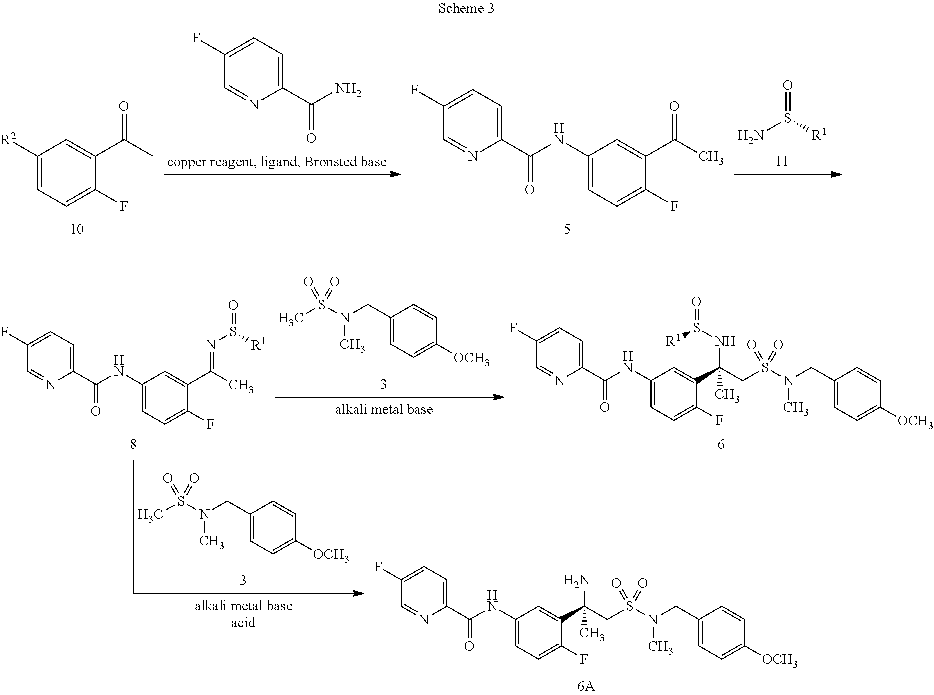

As shown in Scheme 3, coupling of ketone (10) in the presence of a copper reagent, a ligand, and a Brønsted base to provide the ketone (5), which can be condensed with sulfonamide (11) to yield the sulfinyl imine (8). To prepare the PMB-protected sulfonamide (6), sulfinyl imine (8) is reacted with an alkali metalated species of methylsulfonamide (3). Alternatively, to prepare the PMB-protected amine (6A), sulfinyl imine (8) is reacted with an alkali metalated species of methylsulfonamide (3) followed by an acid.

Scheme 4 illustrates one embodiment of an alternative process for preparing the ketone (5).

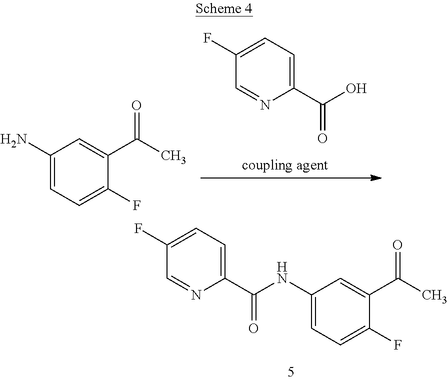

As shown in Scheme 4, ketone (5) is prepared by coupling 1-(5-amino-2-fluorophenyl)ethanone acid with 5-fluoropicolinic acid. Typically, the coupling is carried using a coupling agent such as T₃P, DCC or EDC.

Scheme 5 illustrates another embodiment of an alternative process for preparing the amine (7).

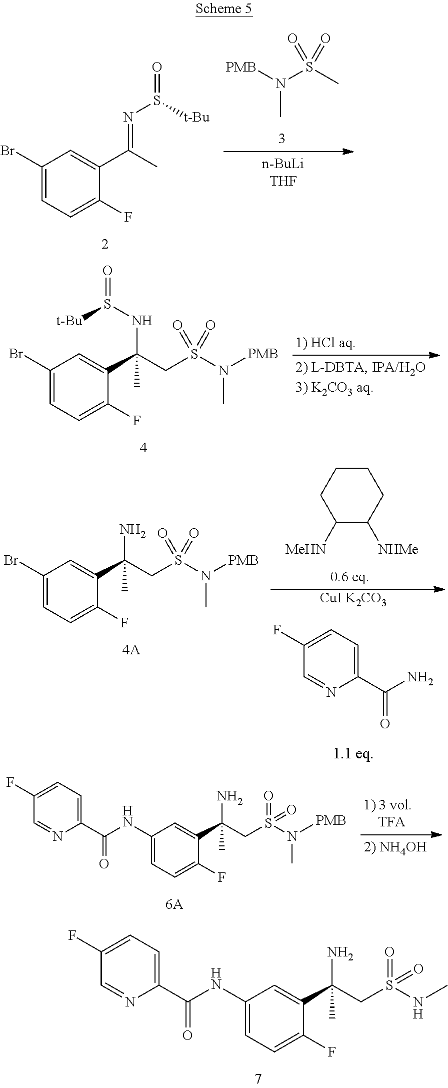

As shown in Scheme 5, the sulfinyl imine (2) is reacted with an alkali-metalated species of the methyl sulfonamide (3) followed by an acid such as tartartic acid to form the aryl fluoride (4). The PMB-protected amine (6A) is prepared by coupling a aryl fluoride (4A) with 5-fluoropicolinamide in the presence of a copper or palladium reagent, a ligand, and a Brønsted base. Suitable ligands include N,N′-dialkyl-containing ligands. Non-limiting examples of such ligands include trans-N,N′-dimethylcyclohexane-1,2-diamine (pictured in Scheme 5 and Example 6) and N,N′-dimethylethylene-1,2-diamine. The PMB-protected amine (6A) is deprotected with an acid such as trifluoroacetic7 acid to provide the amine (7).

### PREPARATIVE EXAMPLES

Methods for preparing the compound of Formula (I) as well as synthetic intermediates useful for its preparation according to the invention are exemplified below. Starting materials are made according to procedures known in the art or as illustrated herein.

Certain starting materials can be prepared according to procedures known in the art. For example, 1-(5-bromo-2-fluorophenyl)ethanone (10a) can be prepared as described in U.S. Patent Application Publication No. 2003/0187026. (R)-2-methylpropane-2-sulfinamide can be prepared as described in as described in Liu, Guangcheng et al., Journal of the American Chemical Society, 119(41), 9913-9914; 1997. 4-Methoxybenzaldehyde can be prepared as described in Adams, Roger et al., Journal of the American Chemical Society, 46, 1518-21; 1924. 1-(5-Amino-2-fluorophenyl)ethanone can be prepared as described in Culbertson, Townley P. et al., Journal of Heterocyclic Chemistry, 24(6), 1509-20; 1987. 5-Fluoropicolinamide can be prepared as described in International Patent Application Publication No. WO 2003/015776. 5-Fluoropicolinic acid can be prepared as described in U.S. Pat. No. 4,798,619.

### Abbreviations

g: grams

mg: milligrams

mL: milliliters

mmol: millimoles

r.t.: room temperature

h: hours

min: minutes

Me: methyl

Et: ethyl

Pr: propyl

iPr: isopropyl

Bu: butyl

tBu: tert-butyl

Ph: phenyl

Bn: benzyl

OAc: acetate

tol: tolyl

Tf: trifluoromethanesulfonyl

PMB: p-methoxybenzyl

Ms: methanesulfonyl

EtOAc: ethyl acetate

THF: tetrahydrofuran

2MeTHF: 2-methyltetrahydrofuran

MeCN: acetonitrile

CPME: cyclopentylmethyl ether

HMDS: hexamethyldisilazane

HMPA: hexamethylphosphoramide

TFA: trifluoroacetic acid

TBAI: tetrabutylammonium iodide

DCC: 1,3-dicyclohexylcarbodiimide

EDC: 1-ethyl-3-(3-dimethylaminopropyl)carbodiimide

T₃P: propylphosphonic anhydride

DMS: dimethylsulfide

TMSOTf: trimethylsilyl trifluoromethanesulfonate

DACH: 1,2-diaminocyclohexane

DMEDA: N,N′-dimethylethylenediamine

DM-DACH: trans-N,N′-dimethylcyclohexane-1,2-diamine

TBAI: tetrabutylammonium iodide

aminobiphenylPdOMs: aminobiphenylmethanesulfonate

Dba: dibenzylideneacetone

TMHD: 2,2,6,6-tetramethyl-3,5-heptanedionate

AcChxn: 2-acetylcyclohexanone

iBuChxn: 2-isobutyrylcyclohexanone

TMEDA: Tetramethylethylenediamine

Rockphos: 2-di(tert-butyl)phosphino-2′,4′,6′-triisopropyl-3-methoxy-6-methylbiphenyl

tBuBrettphos: 2-(di-tert-butylphosphino)-2′,4′,6′-triisopropyl-3,6-dimethoxy-1,1′-biphenyl

AdBrettphos: di((adamantan-1-yl)(2′,4′,6′-triisopropyl-3,6-dimethoxy-[1,1′-biphenyl]-2-yl)phosphine

Xphos: 2-dicyclohexylphosphino-2′,4′,6′-triisopropylbiphenyl

tBu₃P: tri-tert-butyl phosphine

Dtbpf: 1,1′-bis(di-tert-butylphosphino)ferrocene

tBuXPhos: 2-di-tert-butylphosphino-2′,4′,6′-triisopropylbiphenyl

Qphos: 1,2,3,4,5-pentaphenyl-1′-(di-tert-butylphosphino)ferrocene

tBuBippyphos: 5-(di-tert-butylphosphino)-1′,3′,5′-triphenyl-1′H-[1,4′]bipyrazole

Bippyphos: 5-(di-tert-butylphosphino)-1′,3′,5′-triphenyl-1′H-[1,4′]bipyrazole

AdBippyphos (Adamantyl-BippyPhos): 5-[di(1-adamantyl)phosphino]-1′,3′,5′-triphenyl-1′H-[1,4]bipyrazole

Xphos: 2-dicyclohexylphosphino-2′,4′,6′-triisopropylbiphenyl

Sphos: 2-dicyclohexylphosphino-2′,6′-dimethoxybiphenyl

Johnphos: (2-biphenyl)di-tert-butylphosphine

Davephos: 2-dicyclohexylphosphino-2′-(N,N-dimethylamino)biphenyl

RuPhos: 2-dicyclohexylphosphino-2′,6′-diisopropoxybiphenyl

tetramethyl tBuXPhos: 2-di-tert-butylphosphino-3,4,5,6-tetramethyl-2′,4′,6′-triisopropyl-1,1′-biphenyl

Brettphos: 2-(dicyclohexylphosphino)3,6-dimethoxy-2′,4′,6′-triisopropyl-1,1′-biphenyl

cataXCium A: di(1-adamantyl)-n-butylphosphine

AmPhos: di-tert-butyl(4-dimethylaminophenyl)phosphine

tBu₂PBu: di-tert-butyl(n-butyl)phosphine

Xantphos: 4,5-bis(diphenylphosphino)-9,9-dimethylxanthene

Dppf: 1,1′-bis(diphenylphosphanyl) ferrocene

Dippf: 1,1′-bis(di-i-propylphosphino)ferrocene

Dppp: propane-1,3-diylbis(diphenylphosphane)

Dppb: 1,4-bis(diphenylphosphino)butane

DPEPhos: bis[(2-diphenylphosphino)phenyl]methane

BINAP: 2,2′-bis(diphenylphosphino)-1,1′-binaphthyl

Josiphos SL-J009: 1-[(SP)-2-(Dicyclohexylphosphino)ferrocenyl]ethyldi-tert-butylphosphine

MorDalphos: di(1-adamantyl)-2-morpholinophenylphosphine

MeCgPPh=1,3,5,7-tetramethyl-6-phenyl-2,4,8-trioxa-6-phosphaadamantane

cataCXium PtB: N-Phenyl-2-(di-t-butylphosphino)pyrrole

o-Tol₃P: tri(o-tolyl)phosphine

Cy₃P: tricyclohexylphosphane

tBuXPhos: 2-di-tert-butylphosphino-2′,4′,6′-triisopropylbiphenyl

Bipyridine: 2,2′-bipyridine

4,4-di-tBubipy: 4,4′-di-tert-butyl-2,2′-bipyridine

Neocuproine: 2,9-dimethyl-1,10-phenanthroline

Tetramethylphenanthroline: 2,3,4,5-Tetramethyl-1,7-phenanthroline

Terpyridine: 2,6-bis(2-pyridyl)pyridine

tri-tBu-terpyridine: 4,4′,4″-tri-tert-butyl-2,2′:6′,2″-terpyridine

8-hydroxyquinoline: 8-quinolinol

SALOX: 2-hydroxybenzaldehyde oxime

Chxn-Py-Al: N¹,N²-bis(pyridin-2-ylmethylene)cyclohexane-1,2-diamine

TMHD: 2,2,6,6-tetramethyl-3,5-heptanedione

DMPAO: (2,6-dimethylanilino)(oxo)acetic acid

DMeOPAO: 2-((2,6-dimethoxyphenyl)amino)-2-oxoacetic acid

DCF₃PAO: 2-((3,5-bis(trifluoromethyl)phenyl)amino)-2-oxoacetic acid

TBPmalonate: tetrabutylphosphonium malonate (2 phosphonium units)

tBu-TMG: 2-tert-butyl-1,1,3,3-tetramethylguanidine

TMG: N,N,N′,N′-tetramethylguanidine

n-BuLi: n-butyllithium

n-HexLi: n-hexyllithium

The following examples are provided so that the invention may be more fully understood. These examples are illustrative only and should not be construed as limiting the invention in any way.

Nuclear magnetic resonance (NMR) spectra were recorded for ¹H NMR at 500 MHz or 400 MHz. Chemical shifts were reported in ppm relative to the residual deuterated solvent for ¹H. Splitting patterns for ¹H NMR signals are designated as: s (singlet), d (doublet), t (triplet), q (quartet), quint (quintuplet), broad singlet (br s) or m (multiplet).

### Example 1

### Example 1 Describes One Embodiment of the Process Illustrated in Scheme 1

### Step A: Preparation of (R,E)-N-(1-(5-bromo-2-fluorophenyl)ethylidene)-2-methylpropane-2-sulfinamide (2a)

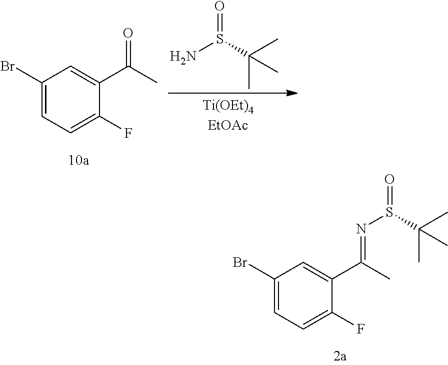

To a reactor (R-1) equipped with a temperature probe, nitrogen inlet and agitator was charged 1-(5-bromo-2-fluorophenyl)ethanone (95%, 9.3 g, 40.7 mmol) and (R)-2-methylpropane-2-sulfinamide (5.4 g, 44.6 mmol). Next, ethyl acetate was charged to the reactor (47 mL). Agitation was begun and the reaction was warmed to 50 to 70° C. titanium (IV) ethoxide (10 mL, 40.7 mmol) was charged to the reaction, and the reaction was allowed to agitate at 50 to 70° C. When the reaction was deemed complete, the reaction was cooled to 20 to 30° C.

In a separate reactor (R-2) equipped with an agitator was charged sodium bicarbonate (6.5 g, 77 mmol) and water (90 mL). The contents of R-2 were agitated until all solids had dissolved. Next, CELITE (10 g) was charged to the vessel. The contents of reactor R-1 were added to reactor R-2. Upon complete addition, the reaction was allowed to agitate at 20 to 30° C. At this point, the reaction was filtered and the filtrate containing 2a was charged into a new reactor (R-3). The aqueous later was separated and extracted with 30 mL ethyl acetate. The organic layers were combined and washed with 30 mL of a saturated aqueous sodium chloride solution. The organic layer was then separated and concentrated to about 20 mL total volume. 80 mL n-heptane was charged to the reactor and concentrated the solution to a total volume of about 70 mL. The concentrate was cooled to 0 to 10° C. and allowed to age. The solids were filtered, washed with 20 mL of a 4:1 ratio of n-heptane to ethyl acetate, and dried in a vacuum oven at 40° C. to provide 2a (9.9 g, 30.9 mmol). ¹H NMR (CDCl₃, 400 MHz) δ 7.77-7.79 (m, 1H), 7.52-7.56 (m, 1H), 7.01-7.06 (m, 1H), 2.77 (d, J=3.2 Hz, 3H), 1.34 (s, 9H); MS (m/z): [M+H]⁺ calcd for C₁₂H₁₆BrFNOS, 322.01; found, 321.94.

### Step B: Preparation of (R)-2-(5-bromo-2-fluorophenyl)-2-((R)-1,1-dimethylethylsulfinamido)-N-(4-methoxybenzyl)-N-methylpropane-1-sulfonamide (4a)

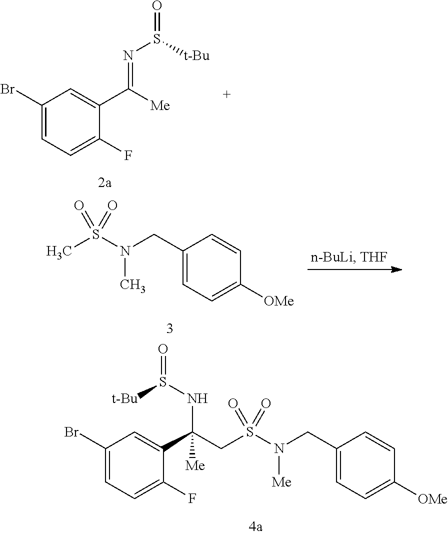 [SMILES: C/C(=N\[S@](=O)C(C)(C)C)c1cc(Br)ccc1F ; COc1ccc(CN(C)S(C)(=O)=O)cc1 ; COc1ccc(CN(C)S(=O)(=O)C[C@](C)(N[S@](=O)C(C)(C)C)c2cc(Br)ccc2F)cc1]

To a reactor (R-1) equipped with a temperature probe, nitrogen inlet, and agitator was charged N-(4-methoxybenzyl)-N-methylmethanesulfonamide 3 (71.6 g, 312 mmol) (Example 2) followed by THF (400 mL). Agitation was begun and the resulting solution was cooled to −15-20° C. n-BuLi (2.5 M in hexanes, 125 mL, 312 mmol) was then added at a sufficient rate to maintain the internal temperature. After 30 min, the reaction was cooled to −35-45° C.

To a second reactor (R-2) equipped with a temperature probe, nitrogen inlet, and agitator was charged (R,E)-N-(1-(5-bromo-2-fluorophenyl)ethylidene)-2-methylpropane-2-sulfinamide 2 (50 g, 156 mmol) followed by THF (100 mL). Agitation was begun and the resulting solution was added over 2 h to R-1. When the reaction in R-1 was deemed complete, it was quenched with water (500 mL) and warmed to ambient temperature. To the biphasic mixture was added 10% aqueous NaCl (250 mL) and the product was extracted twice with CH₂Cl₂ (2×500 mL). The organic layers were combined, dried over MgSO₄, filter, and concentrated. The crude product was purified by silica gel chromatography to afford 4a (45.4 g, 82.4 mmol). ¹H NMR (CDCl₃, 400 MHz) δ 7.68 (dd, J=1.9, 5.7 Hz, 1H), 7.41-7.45 (m, 1H), 7.20-7.24 (m, 2H), 6.99 (dd, J=6.9, 9.7 Hz, 1H), 6.84-6.89 (m, 2H), 5.89 (s, 1H), 4.19 (s, 2H), 3.82-3.92 (m, 2H), 3.80 (s, 3H), 2.71 (s, 3H), 1.92 (s, 3H), 1.37 (s, 9H); MS (m/z): [M+H]⁺ calcd for C₂₂H₃₀BrFN₂O₄S₂, 551.09; found, 550.93.

### Step C: Preparation of (R)-2-((R)-1,1-dimethylethylsulfinamido)-2-(2-fluoro-5-fluoropicolinamide)phenyl)-N-(4-methoxybenzyl)-N-methylpropane-1-sulfonamide (6a)

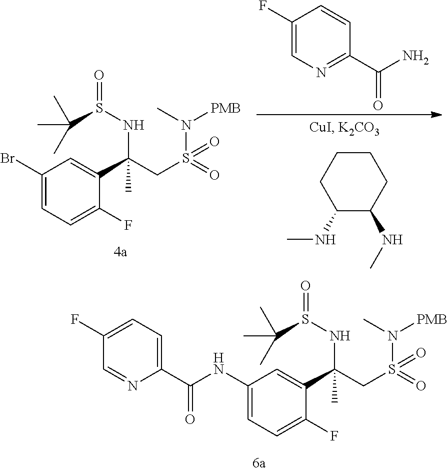

To a reactor (R-1) equipped with a temperature probe, nitrogen inlet and agitator was charged 5-fluoro-2-pyridinecarboxamide (1.049 g, 7.49 mmol), K₂CO₃ (2.82 g, 20.42 mmol), trans-N,N′-dimethylcyclohexane-1,2-diamine (2.147 mL, 13.61 mmol), copper(I) iodide (1.296 g, 6.81 mmol), toluene (8.5 mL) and 4a (37 wt % in toluene, 10 g, 6.81 mmol). Agitation was begun and the reaction was warmed to 80-90° C. When the reaction was deemed complete, the reaction was cooled to 20 to 30° C. In a separate reactor (R-2) equipped with an agitator was charged a 5% sodium chloride solution. The aqueous layer was separated and the organic layer was washed with 5% sodium chloride solution until the aqueous layer was clear. The organic layer was then concentrated to provide 6a (3.09 g, 5.08 mmol). ¹H NMR (CDCl₃, 500 MHz) δ 10.79 (s, 1H), 8.74 (d, J=2.9 Hz, 1H), 8.24 (dd, J=4.2, 8.5 Hz, 2H), 7.98 (dt, J=2.8, 5.5 Hz, 1H), 7.92 (dq, J=2.4, 8.8 Hz, 1H), 7.17-7.23 (m, 2H), 6.90-6.95 (m, 2H), 5.58 (s, 1H), 4.00-4.10 (m, 2H), 3.81-3.90 (m, 2H), 3.74 (s, 3H), 2.56 (s, 3H), 1.94 (s, 3H), 1.20 (s, 9H); MS (m/z): [M+H]⁺ calcd for C₂₈H₃₅F₂N₄O₅S₂, 609.20; found, 609.10.

### Step D: Preparation of (R)—N-(3-(2-amino-1-(N-methylsulfamoyl)propan-2-yl)-4-fluorophenyl)-5-fluoropicolinamide (7)

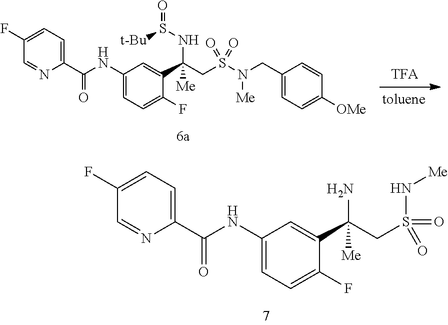 [SMILES: COc1ccc(CN(C)S(=O)(=O)C[C@](C)(N[S@](=O)C(C)(C)C)c2cc(NC(=O)c3ccc(F)cn3)ccc2F)cc1 ; CNS(=O)(=O)C[C@](C)(N)c1cc(NC(=O)c2ccc(F)cn2)ccc1F]

To a reactor (R-1) equipped with a temperature probe, nitrogen inlet, and agitator was charged (R)-2-((S)-1,1-dimethylethylsulfinamido)-2-(2-fluoro-5-fluoropicolinamide)phenyl)-N-(4-methoxybenzyl)-N-methylpropane-1-sulfonamide (6a) (24.1 g, 39.6 mmol) followed by toluene (121 mL). Agitation was begun, and then TFA (15.3 mL, 198 mmol) was charged. The resulting homogeneous solution was heated to 55-65° C. and agitated until the reaction was deemed complete. The reaction was cooled to ambient temperature and the product was extracted three times with water (3×100 mL). The combined aqueous layers were basified with 20% aqueous Na₂CO₃ to pH 10 and the product extracted three times with CH₂Cl₂ (3×300 mL). The combined organic layers were dried over MgSO₄, filtered, and concentrated to afford 7 (8.1 g, 21.1 mmol). ¹H NMR (DMSO, 400 MHz) δ 10.57 (s, 1H), 8.73 (d, J=2.8 Hz, 1H), 8.24 (dd, J=4.8, 8.8 Hz, 1H), 8.15 (dd, J=2.5, 7.6 Hz, 1H), 7.98 (dt, J=2.8, 8.6 Hz, 1H), 7.85-7.90 (m, 1H), 7.11 (dd, J=8.7, 12.0 Hz, 1H), 6.81 (s, 1H), 3.63 (d, J=14.0 Hz, 1H), 3.45 (d, J=14.0 Hz, 1H), 2.50 (s, 3H), 1.53 (s, 3H); MS (m/z): [M+H]⁺ calcd for C₁₆H₁₉F₂N₄O₃S, 385.11; found, 385.02.

### Step E: Preparation of Verubecestat (I)

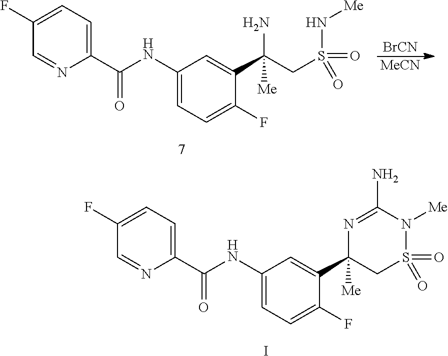

To a reactor (R-1) equipped with a temperature probe, nitrogen inlet, and agitator was charged (R)—N-(3-(2-amino-1-(N-methylsulfamoyl)propan-2-yl)-4-fluorophenyl)-5-fluoropicolinamide (7) (8.10 g, 21.1 mmol) followed by MeCN (40.5 mL). Agitation was begun and then BrCN (5 M in MeCN, 6.32 mL, 31.6 mmol) was charged. The slurry was heated to 80-90° C. and the resulting homogeneous solution was agitated until the reaction was deemed complete. The reaction was cooled to ambient temperature and diluted with EtOAc (122 mL). The organics were washed twice with saturated aqueous NaHCO₃ (2×80 mL), dried over MgSO₄, filtered, and concentrated. The solids were dissolved in EtOAc (52 mL) and heated to 40-50° C. Heptane (8.6 mL) was charged followed by a small amount of crystalline seeds of I. Additional heptane (69 mL) was added over 4 h and the slurry cooled to ambient temperature. The solids were collected, washed with heptane (26 mL), and dried under vacuum with a N₂ sweep to afford I (2.89 g, 7.06 mmol). ¹H NMR (CDCl₃, 400 MHz) δ 9.69 (s, 1H), 8.20-8.32 (m, 2H), 7.99 (m, 1H), 7.52-7.66 (m, 2H), 7.07 (m, 1H), 5.33 (s, 2H), 3.97 (d, J=13.9 Hz, 1H), 3.67 (d, J=14.0 Hz, 1H), 3.22 (s, 3H), 1.79 (s, 3H); MS (m/z): [M+H]⁺ calcd for C₁₇H₁₈F₂N₅O₅S, 410.11; found, 410.03.

### Example 2

### Preparation of N-(4-methoxybenzyl)-N-methylmethanesulfonamide

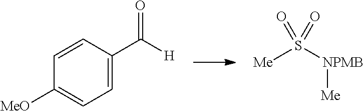

To a reactor (R1) equipped with a temperature probe, nitrogen inlet, and agitator was charged MeOH (92 kg) and 4-methoxybenzaldehyde (230.0 kg, 1689 mol). Agitation of R1 was begun and the internal temperature adjusted to 0° C. A solution of methylamine (30% in EtOH, 209.8 kg, 2026 mol) was charged to R1 dropwise over 6 h. The internal temperature of R1 was then adjusted to 20° C. and the mixture agitated until the condensation was judged to be complete, at which point the internal temperature was adjusted to 0° C. A second reactor (R2) was charged with THF (206 kg) followed by NaBH₄ (51.2 kg, 1351 mol). Agitation was begun and the reaction mixture from R1 was transferred to R2 over 8 h. The mixture was agitated until the reduction was judged to be complete. A third reactor (R3) was charged with water (115 kg) and 35% aqueous HCl (404 kg). Agitation of R3 was begun and the internal temperature was adjusted to 0° C. The reaction mixture from R2 was transferred to R3 over 12 h. The mixture was agitated until the reduction was judged to be complete. CH₂Cl₂ (969 kg) was charged to R3 followed by 50% aqueous NaOH (366 kg) over 6 h, at which point the internal temperature was adjusted to 20° C. The resulting solids were separated and washed with CH₂Cl₂ (157 kg) and the filtrate was transferred to R3. The layers were allowed to separate and the organic layer concentrated to approximately 1-2 volumes. CH₂Cl₂ (1220 kg) was charged to R3 and the contents concentrated; this process was repeated until the amount of residual water was judged to be satisfactory. Triethylamine (243 kg, 2400 mol) was charged to R3 and the contents of R3 transferred to a fourth reactor (R4). To R3 was charged methanesulfonyl chloride (223 kg, 1947 mol) and CH₂Cl₂ (635 kg), agitation was begun, and the internal temperature was adjusted to 0° C. The mixture in R4 was transferred to R3 dropwise over 12 h and then R3 was further agitated for 6 h. Water (572 kg) was then charged to R3 and the internal temperature adjusted to 20° C. The layers were allowed to separate and the organic layer washed twice with 2% aqueous NaCl (564 kg). The organic layer was concentrated under reduced pressure below 25° C. and then heptane (30 kg) was charged dropwise, followed by seed crystals (8 g) and additional heptane (1517 kg) over 20 h. The resulting slurry was agitated for 8 h before the solids were collected and washed with 10% CH₂Cl₂ in heptane (320 kg) to provide N-(4-methoxybenzyl)-N-methylmethanesulfonamide (290.8 kg).

### Example 3

### Preparation of N-(3-acetyl-4-fluorophenyl)-5-fluoropicolinamide (5)

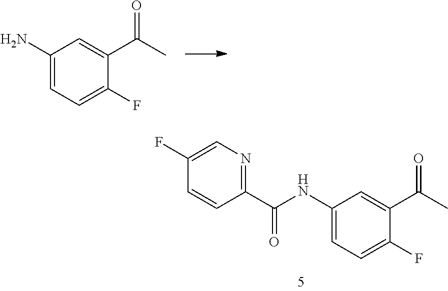

A round bottom flask equipped with an agitator was charged with 1-(5-amino-2-fluorophenyl)ethanone (1 g, 6.53 mmol), 5-fluoropicolinic acid (1.1 g, 7.80 mmol), THF (10 ml) and Hunig's Base (3.4 ml, 19.47 mmol). The reaction mixture was cooled to −10° C. and T₃P 50 wt % in 2-MeTHF (5.82 g, 9.14 mmol) was added slowly. The mixture is stirred at r.t. for 1 hour. Then the reaction mixture was cooled to 0° C. and water was added until a slurry was formed. The slurry was then filtered and rinsed with water to produce 1.80 g of N-(3-acetyl-4-fluorophenyl)-5-fluoropicolinamide.

### Example 4

### Example 4 Describes One Embodiment of the Process Illustrated in Scheme 2

### Step A: Preparation of Amine (9a)

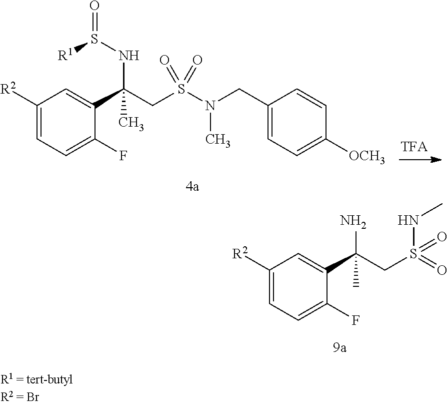

To a reactor (R1) equipped with a temperature probe, nitrogen inlet, and agitator was charged 4a (1 equiv). Toluene was charged to R1 and agitation was begun. Trifluoroacetic acid (10 equiv) was charged to R1 and the mixture heated to 60° C. until the reaction was judged complete. The contents were cooled to ambient temperature, at which point water was charged to R1. The layers were allowed to separate and the aqueous layer transferred to a second reactor (R2). The pH of the solution in R2 was adjusted to greater than 10 using a basic aqueous solution and the contents extracted three times with dichloromethane. The combined organic layers were dried over MgSO₄, filtered, and concentrated to afford 9a.

### Step B: Preparation of Amine (7)

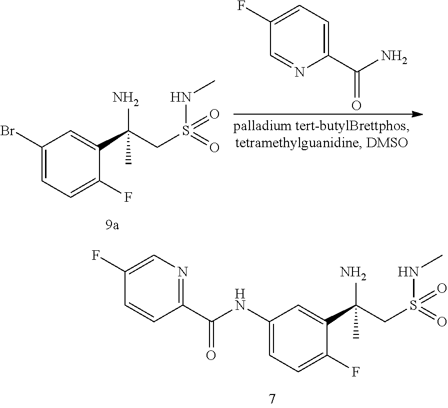 [SMILES: CNS(=O)(=O)C[C@](C)(N)c1cc(Br)ccc1F ; NC(=O)c1ccc(F)cn1 ; CNS(=O)(=O)C[C@](C)(N)c1cc(NC(=O)c2ccc(F)cn2)ccc1F]

This description provided for this step is prophetic.

To reactor (R1) equipped with a temperature probe, nitrogen inlet, and agitator is charged 9a (1 equiv), 2-fluoropicolinamide (1.1 equiv), and palladium tert-butylBrettPhos G3 precatalyst (0.03 equiv). Dimethylsulfoxide and 1,1,3,3-tetramethylguanidine (3 equiv) are charged to R1 and agitation is begun. The mixture is heated to 70° C. until the reaction is judged complete. The contents are cooled to ambient temperature, at which point ethyl acetate is charged and the mixture is washed three times with water. The organic layer is dried over MgSO₄, filtered, and concentrated to afford 7.

### Example 5

### Example 5 Describes One Embodiment of the Process Illustrated in Scheme 3

### Step A: Preparation of Amine (8a) (wherein R¹ is tert-butyl) from Ketone (5)

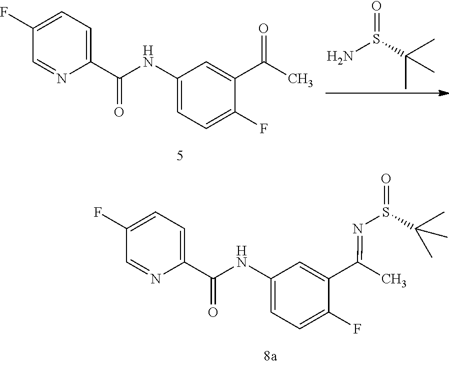

To a reactor (R1) equipped with a temperature probe, nitrogen inlet, and agitator was charged 5 (1 equiv), titanium ethoxide (2 equiv), and 2-methyl-2-propanesulfinamide (1.4 equiv). Cyclopentylmethyl ether was charged to R1 and agitation was begun. The mixture was heated until the reaction was judged complete. The contents were cooled to ambient temperature, at which point water was charged and the solids were filtered off. The organic layer from the filtrate was collected and dried over MgSO₄, filtered, and concentrated to afford 8a.

### Step B: Preparation of PMB-Protected Sulfonamide (6)

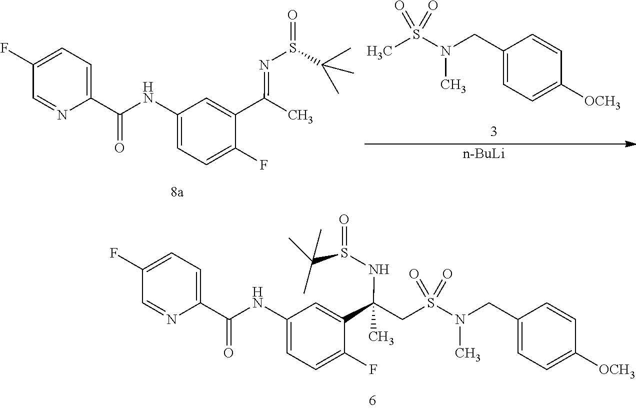

To a reactor (R1) equipped with a temperature probe, nitrogen inlet, and agitator was charged 3 (1.75 equiv) followed by THF. Agitation was begun and the resulting solution was cooled to −15-20° C. n-BuLi (2.5 M in hexanes, 1.75 equiv) was then added at a sufficient rate to maintain the internal temperature. After 30 min, the reaction was cooled to −35-45° C. To a second reactor (R2) equipped with a temperature probe, nitrogen inlet, and agitator was charged 8a (1.00 equiv). Agitation was begun and the resulting solution was added over 2 h to R1. When the reaction in R1 was deemed complete, it was quenched with water and warmed to ambient temperature. To the biphasic mixture was added 10% aqueous NaCl and the product was extracted twice with CH₂Cl₂. The organic layers were combined, dried over MgSO₄, filter, and concentrated. The crude product was purified by silica gel chromatography to afford 6.

### Example 6

### Example 6 Describes One Embodiment of the Process Illustrated in Scheme 5

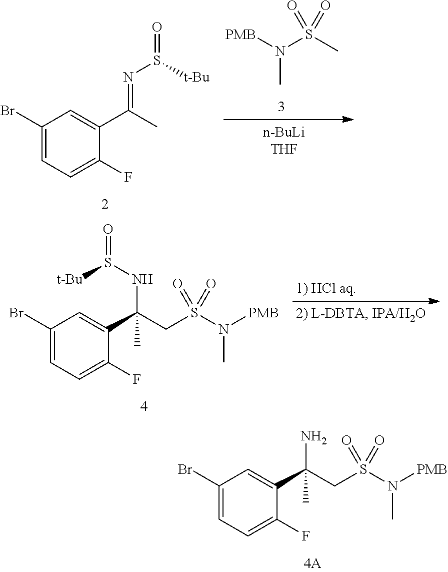

To a reactor (R1) equipped with a temperature probe, nitrogen inlet, and agitator was charged 3 (272 g, 1.36×) followed by THF. The temperature of R1 was adjusted to −20 to −15° C. nBuLi (2.27M, 1.5 to 1.6×) was added dropwise to R1 over 2 h at a rate to maintain the internal temperature of the reaction at −20 to −15° C. The batch was then agitated at −20 to −15° C. for 0.5 to 1 h. The temperature of R1 was adjusted to −70 to −60° C. To a reactor (R2) equipped with a temperature probe, nitrogen inlet, and agitator was charged 2 (200 g, 1×) followed by THF. The batch in R2 was agitated for 0.5 h at 25-30° C. Add the reaction mixture in R2 into R1 at −70 to −60° C. over 2 h. The resulting reaction mixture in R1 was stirred at −70° C. to −60° C. for 0.5 to 1 h. When the reaction in R1 was deemed complete, the reaction mixture was quenched by adding a solution of AcOH (75 g, 0.37-0.40×) in THF (18 g, 0.05-0.1×) to R1 at −70° C. to −60° C. in 1 h. The temperature of R1 was adjusted to 15 to 25° C. 232.4 g of 4 was obtained by assay. 500 g of 6N HCl aqueous solution was charged to R1 and agitated at 15-25° C. for 1 to 2 h. When the reaction was deemed complete, ethyl acetate (640 g, 3-3.5×) and 600 g (3-3.5×) pure water was charged into R1. The aqueous layer was separated and removed. 10% aqueous K₂CO₃ solution (1.4 kg, 6.5 to 7.5×) was charged to R1 within 0.5 h (pH of aqueous layer was 6 to 7). The organic layer was washed with pure water (700 g, 3-4×) and the aqueous layer was removed. The organic phase was concentrated under vacuum at 40° C. to 50° C., co-distilling with 2-propanol under vacuum at 40 to 50° C. until the residual THF and ethyl acetate in the resulting solution (3.0 to 4.0×) was <0.05% (2.3 kg of 2-propanol was used in the azeotropic removal of THF and ethyl acetate). Additional 2-propanol was charged to R1 (540 g (2.0 to 4.0×) followed by dibenzoyl-L-tartaric acid (90.7 g, 0.45-0.50×). Pure water was then charged to R1 (820 g, 4.0-4.5×) and R1 was warmed to 65° C. for 0.5 h. The batch was cooled to 50° C. and seeded. The batch in R1 was further cooled to 5 to 15° C. over 4 h and aged for 10-20 h. The batch was filtered and the wet cake was slurried with 400 g (1.5-2.5×) 2-propanol/water (v/v 3:2). The wet cake was washed with pure water twice (600 g, 3.0-4.0×). The wet cake was transferred to R1 followed by toluene (870 g, 4.3 to 4.5×). 30% K₂CO₃ aqueous solution (1 kg, 5.0 to 6.0×) was charged to R1 and the batch was stirred for 0.5 h at 20 to 30° C. The aqueous layer was separated and 4A was carried forward as a toluene solution.

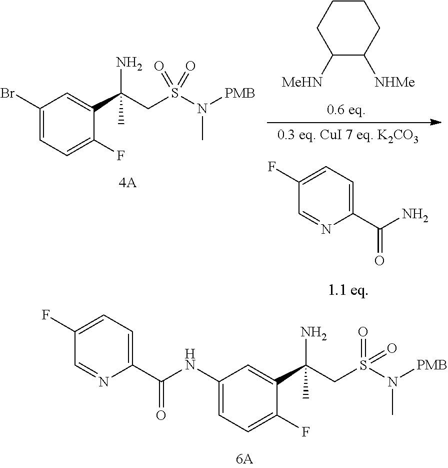

To a reactor (R1) equipped with a temperature probe, nitrogen inlet and agitator was charged 5-fluoropicolinamide (11.93 g), K₂CO₃ (74.9 g), trans-N,N′-dimethylcyclohexane-1,2-diamine (6.6 g), copper(I) iodide (4.42 g), toluene (150 g) and 4a (34 wt % in toluene, 34.5 g). The mixture was sparged with argon for 2 h to remove oxygen. Agitation was begun and the reaction was warmed to 80-90° C. The reaction was quenched by adding 13.9 g ethane-1,2-diamine and stirred for 0.5 h. The organic layer was washed with AcOH (27.86 g) and water (172 g) followed by 7% NaHCO₃ solution. The organic layer was then concentrated to provide 6A. ¹H NMR (CDCl₃, 400 MHz) δ 9.97 (s, 1H), 8.55 (d, 2H), 8.42 (dd, 1 h), 8.01 (m, 2H), 7.65 (m, 1H), 7.29 (m, 3H), 7.23 (m, 6H), 6.93 (d, 2H), 4.21 (m, 1H), 3.97 (m, 1H), 3.89 (s, 3H), 3.48 (d, 1H), 2.66 (s, 3H), 1.77 (s, 3H).

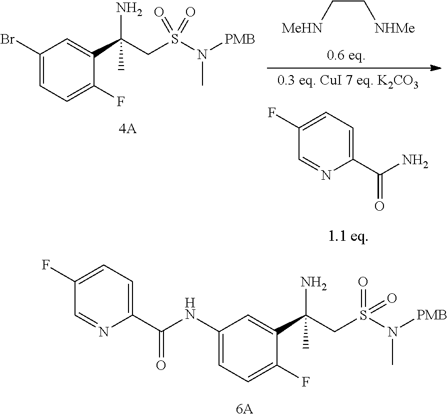

The following describes an alternate procedure for the synthesis of 6A from 4A using or N,N′-dimethylethylenediamine (DMEDA). To an autoclave reactor (R1) equipped with mechanical stirror, temperature probe, 4A solution in toluene (178.66 g×19.6%=35 g 4A), 5-fluoropicolinamide (12.1 g) and K₂CO₃ aqueous solution (76 g K₂CO₃, in 105 g water), were charged, followed by 4.16 g DMEDA. The mixture was sparged with argon for 2 h to remove oxygen. Then, 4.49 g CuI was charged and the mixture was sparged with argon for 1 hour. The autoclave was then sealed and heated to 105° C. (internal temperature) for 24 h with agitation. The reaction was quenched by adding 14.15 g ethane-1,2-diamine and stirred for 0.5 h. The organic layer was washed with AcOH (28.3 g) in water (172 g) followed by 7% NaHCO₃ solution. The organic layer was then concentrated to provide 6A.

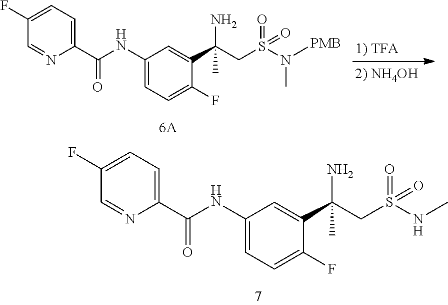

To a reactor (R1) equipped with a temperature probe, nitrogen inlet and agitator was charged 6A (49.9 g) and trifluoroacetic acid (271 g, 5.4×). The mixture was stirred at 55 to 65° C. for 3 h. The temperature of the batch was adjusted to 15 to 25° C. and stirred for 1 to 2 h. Glycolic acid (80 g) and water (320 g) were charged to R2 and the mixture was stirred at 15 to 25° C. until the mixture became clear biphasic solution. The batch in R2 was charged into R1 and stirred at 15 to 25° C. for 1 to 2 h. The layers in R1 were then separated. The aqueous layer of R1 was added dropwise to a reactor (R3) containing NH₄OH (450 g) at 20 to 30° C. over 6 to 8 h. The mixture was then stirred at 15 to 25° C. for 15 to 20 h, filtered and the wet cake washed with water three times, then dried under reduced pressure at 40 to 50° C. for 30 to 40 h to obtain 7. ¹H NMR (DMSO, 400 MHz) δ 10.57 (s, 1H), 8.73 (d, J=2.8 Hz, 1H), 8.24 (dd, J=4.8, 8.8 Hz, 1H), 8.15 (dd, J=2.5, 7.6 Hz, 1H), 7.98 (dt, J=2.8, 8.6 Hz, 1H), 7.85-7.90 (m, 1H), 7.11 (dd, J=8.7, 12.0 Hz, 1H), 6.81 (s, 1H), 3.63 (d, J=14.0 Hz, 1H), 3.45 (d, J=14.0 Hz, 1H), 2.50 (s, 3H), 1.53 (s, 3H); MS (m/z): [M+H]⁺ calcd for C₁₆H₁₉F₂N₄O₃S, 385.11; found, 385.02.

1. A process for the preparation of a compound of Formula (I): 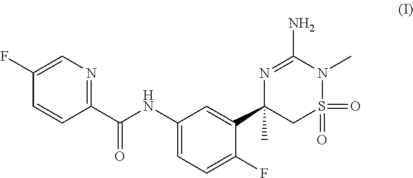 [SMILES: CN1C(N)=N[C@](C)(c2cc(NC(=O)c3ccc(F)cn3)ccc2F)CS1(=O)=O]
comprising reacting amine of the Formula (7): 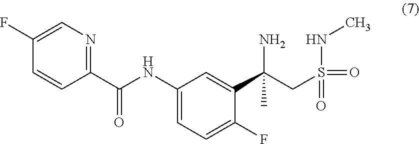 [SMILES: CNS(=O)(=O)C[C@](C)(N)c1cc(NC(=O)c2ccc(F)cn2)ccc1F]
with a cyanating agent to form the compound of Formula (I).

2. The process of claim 1, wherein the cyanating agent is selected from the group consisting of cyanogen, cyanogen bromide, cyanogen fluoride, cyanogen chloride, cyanogen iodide, 2-methoxyphenyl cyanate, 4-methoxyphenyl cyanate, 4-phenylphenyl cyanate, and bisphenol A cyanate.

3. The process of claim 2, wherein the cyanating agent is cyanogen bromide.

4. The process of claim 1, wherein the amine (7) is prepared by deprotecting a PG-protected sulfonamide of Formula (6): 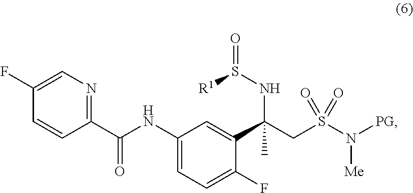
wherein R₁ is:
C₁-C₆ alkyl; or
phenyl, wherein the phenyl is unsubstituted or substituted by 1 to 3 substituents selected from the group consisting of C₁-C₄ alkyl, C₁-C₄ haloalkyl, —O—C₁-C₄ alkyl, —O—C₁-C₄ haloalkyl, halo, and nitro; and
PG is a protecting group;
with trifluoroacetic acid or methane sulfonic acid, to form the amine (7).

5. The process of claim 4 wherein the amine (7) is further purified by:
reacting the amine (7) with an enantiomerically pure chiral acid of Formula A-H to form a diastereomeric salt mixture; separating the salt of the Formula (7A): 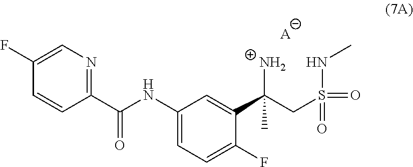
from the diastereomeric salt mixture; reacting the salt (7A) with an aqueous base; and recovering the free base of amine (7),
wherein the chiral acid of Formula A-H is selected from the group consisting of L-tartaric acid, L-(+)-mandelic acid, L-(−)-malic acid, (1S)-(+)-10-camphorsulfonic acid, (−)-di-O,O-p-toluyl-L-tartaric acid, (−)-O,O-dibenzoyl-L-tartaric acid, (+)-camphoric acid, L-pyroglutamic acid, (1S)-(−)-camphanic acid, L-valine, (1S)-(+)-3-bromocamphor-10-sulfonic acid hydrate, L-histidine, D-tartaric acid, D-(−)-mandelic acid, D-(+)-malic acid, (1R)-(−)-10-camphorsulfonic acid, (+)-Di-O,O-p-toluyl-D-tartaric acid, (+)-O,O-dibenzoyl-D-tartaric acid, (−)-camphoric acid, D-pyroglutamic acid, (1R)-(+)-camphanic acid, D-valine, (+)-naproxen, and L-isoleucine.

6. The process of claim 5, wherein the PG-protected sulfonamide (6) is prepared by
coupling an aryl fluoride of Formula (4): 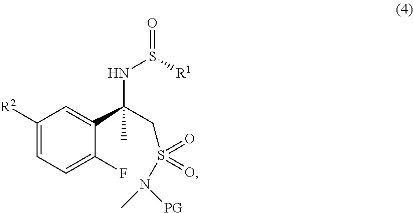
wherein
R₁ is:
C₁-C₆ alkyl; or
phenyl, wherein the phenyl is unsubstituted or substituted by 1 to 3 substituents selected from the group consisting of C₁-C₄ alkyl, C₁-C₄ haloalkyl, —O—C₁-C₄ alkyl, —O—C₁-C₄ haloalkyl, halo, and nitro;
R² is:
halo, selected from the group consisting of bromo, chloro and iodo;
a group of the formula —O—S(O)₂—R^{2a}, wherein R^{2a} is methyl, chloromethyl, dichloromethyl, phenyl, p-trifluoromethylbenzyl, p-toluenyl, p-bromophenyl, p-fluorophenyl, p-methoxyphenyl, 2-nitrophenyl, 4-nitrophenyl, and 2,4-dichlorophenyl; or
a diazonium group; and
PG is a protecting group;
with 5-fluoropicolinamide in the presence of:
a copper or palladium reagent;
a ligand; and
a Brønsted base to form the PG-protected sulfonamide (6).

7. The process of claim 6, wherein the copper or palladium reagent is selected from the group consisting of CuI, CuI-TBAI, CuBr, CuPF₆(MeCN)₄, CuBr₂, [Cu(OTf)]₂-tol, CuCl, Cu metal, Cu₂O, Cu(OAc)₂, (aminobiphenyl)PdOMs dimer, and (aminobiphenyl)PdCl dimer.

8. The process of claim 6, wherein the ligand is selected from the group consist of N,N′-dimethyl diaminocyclohexane, diaminocyclohexane, tBuBrettphos, DMEDA, Xphos, RuPhos, Sphos, water-soluble Sphos, tBuXPhos, Rockphos, Brettphos, AdBrettphos, Qphos, MorDalphos, Amphos, CataCXiumA, tBu₃P, Cy₃P, MeCgPPh, o-tol₃P, PPh₃, BINAP, dppf, dtbpf, Josiphos SL-J009, Johnphos, Xantphos, and NiXantphos.

9. The process of claim 6, wherein the Brønsted base is selected from the group consisting of potassium carbonate, potassium phosphate, cesium carbonate, and potassium bicarbonate.

10. The process of claim 6, wherein the aryl fluoride (4) is prepared by treating a methylsulfonamide of Formula (3): 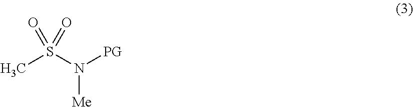
wherein PG is a protecting group with an alkali metal base to form the alkali metalate species of the methylsulfonamide (3); and reacting the alkali metalate species of the methylsulfonamide (3) with sulfinyl imine of Formula (2): 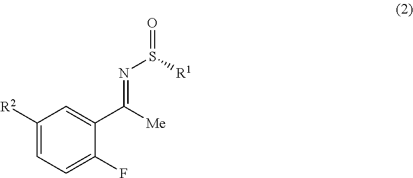
to form the aryl fluoride (4).

11. The process of claim 10, wherein the sulfinyl imine (2) is prepared by: condensing a ketone of Formula (10): 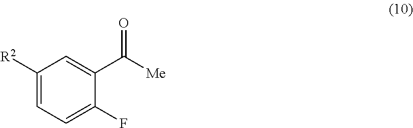
wherein R² is:
(i) halo, selected from the group consisting of bromo, chloro and iodo;
(ii) a group of the formula —O—S(O)₂—R^{2a}, wherein R^{2a} is methyl, chloromethyl, dichloromethyl, phenyl, p-trifluoromethylbenzyl, p-toluenyl, p-bromophenyl, p-fluorophenyl, p-methoxyphenyl, 2-nitrophenyl, 4-nitrophenyl, and 2,4-dichlorophenyl; or
(iii) a diazonium group;
with a sulfinamide of the formula (11):
R¹—S(O)—NH₂ (11)
wherein R₁ is:
C₁-C₆ alkyl; or
phenyl, wherein the phenyl is unsubstituted or substituted by 1 to 3 substituents selected from the group consisting of C₁-C₄ alkyl, C₁-C₄ haloalkyl, —O—C₁-C₄ alkyl, —0-C₁-C₄ haloalkyl, halo, and nitro,
in the presence of a tetra(C₁-C₆ alkoxy)titanium (IV) or tetra(C₁-C₆ alkoxy)zirconium (IV) catalyst to form sulfinyl imine (2).

12. The process of claim 11 wherein R¹ is tert-butyl.

13. The process of claim 12, wherein R² is bromo.

14. A process for preparing an amine of Formula (7): 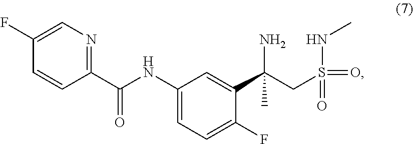 [SMILES: CNS(=O)(=O)C[C@](C)(N)c1cc(NC(=O)c2ccc(F)cn2)ccc1F]
comprising reacting a PG-protected sulfonamide of Formula (6): 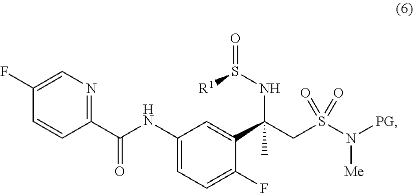
wherein
R₁ is
C₁-C₆ alkyl; or
phenyl, wherein the phenyl is unsubstituted or substituted by 1 to 3 substituents selected from the group consisting of C₁-C₄ alkyl, C₁-C₄ haloalkyl, —O—C₁-C₄ alkyl, —O—C₁-C₄ haloalkyl, halo, and nitro; and
PG is a protecting group;
with trifluoroacetic acid or methane sulfonic acid to form the amine (7).

15. The process of claim 14, wherein the PG-protected sulfonamide (6) is prepared by
coupling an aryl fluoride of Formula (4): 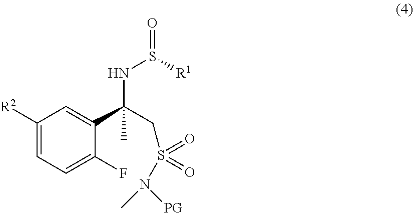
wherein
R₁ is C₁-C₆ alkyl; or phenyl, wherein the phenyl is unsubstituted or substituted by 1 to 3 substituents selected from the group consisting of C₁-C₄ alkyl, C₁-C₄ haloalkyl, —O—C₁-C₄ alkyl, —O—C₁-C₄ haloalkyl, halo, and nitro;
R² is:
halo, selected from the group consisting of bromo, chloro and iodo;
a group of the formula —O—S(O)₂—R^{2a}, wherein R^{2a} is methyl, chloromethyl, dichloromethyl, phenyl, p-trifluoromethylbenzyl, p-toluenyl, p-bromophenyl, p-fluorophenyl, p-methoxyphenyl, 2-nitrophenyl, 4-nitrophenyl, and 2,4-dichlorophenyl; or
a diazonium group; and
PG is a protecting group;
with 5-fluoropicolinamide in the presence of:
a copper or palladium reagent; a ligand; and
a Brønsted base to form the PG-protected sulfonamide (6).

16. A process preparing a PG-protected sulfonamide of Formula (6): 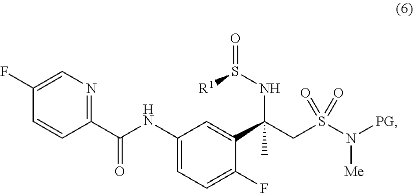
wherein
R₁ is
C₁-C₆ alkyl; or
phenyl, wherein the phenyl is unsubstituted or substituted by 1 to 3 substituents selected from the group consisting of C₁-C₄ alkyl, C₁-C₄ haloalkyl, —O—C₁-C₄ alkyl, —O—C₁-C₄ haloalkyl, halo, and nitro; and
PG is a protecting group;
comprising coupling
an aryl fluoride of Formula (4) 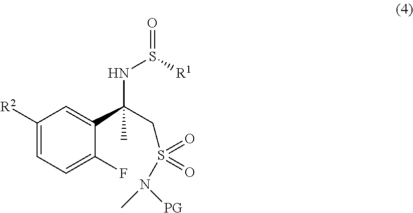
wherein
R₁ is
C₁-C₆ alkyl; or
phenyl, wherein the phenyl is unsubstituted or substituted by 1 to 3 substituents selected from the group consisting of C₁-C₄ alkyl, C₁-C₄ haloalkyl, —O—C₁-C₄ alkyl, —O—C₁-C₄ haloalkyl, halo, and nitro;
R² is:
halo, selected from the group consisting of bromo, chloro and iodo;
a group of the formula —O—S(O)₂—R^{2a}, wherein R^{2a} is methyl, chloromethyl, dichloromethyl, phenyl, p-trifluoromethylbenzyl, p-toluenyl, p-bromophenyl, p-fluorophenyl, p-methoxyphenyl, 2-nitrophenyl, 4-nitrophenyl, and 2,4-dichlorophenyl; or
a diazonium group; and
PG is a protecting group;
with 5-fluoropicolinamide in the presence of a copper or palladium reagent; a ligand; and a Brønsted base to form the PG-protected sulfonamide (6).

17. The process of claim 16, wherein the aryl fluoride (4) is prepared by treating a methylsulfonamide 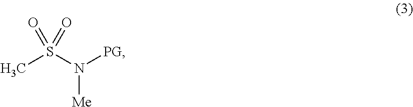
wherein PG is a protecting group, with an alkali metal base to form the alkali metalate species of the methylsulfonamide (3); and reacting the alkali metalate species of the methylsulfonamide (3) with a sulfinyl imine 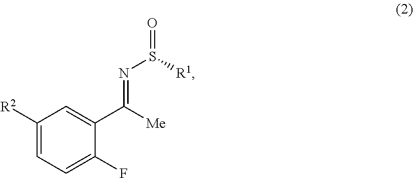
wherein R₁ is C₁-C₆ alkyl; or phenyl, wherein the phenyl is unsubstituted or substituted by 1 to 3 substituents selected from the group consisting of C₁-C₄ alkyl, C₁-C₄ haloalkyl, —O—C₁-C₄ alkyl, —O—C₁-C₄ haloalkyl, halo, and nitro; and
R² is halo, selected from the group consisting of bromo, chloro and iodo; a group of the formula —O—S(O)₂—R^{2a}, wherein R^{2a} is methyl, chloromethyl, dichloromethyl, phenyl, p-trifluoromethylbenzyl, p-toluenyl, p-bromophenyl, p-fluorophenyl, p-methoxyphenyl, 2-nitrophenyl, 4-nitrophenyl, and 2,4-dichlorophenyl; and a diazonium group; to form the aryl fluoride (4).

18. The process of claim 17, wherein the protecting group moiety of PG of said methylsulfonamide 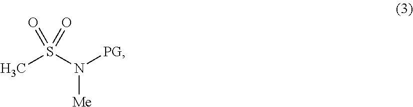
is PMB.

19. The process of claim 1, wherein the amine (7) is prepared by deprotecting a PG-protected amine of Formula (6A): 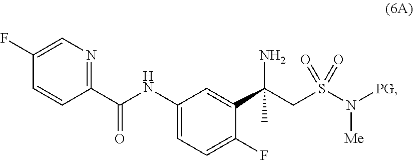
wherein PG is a protecting group, with trifluoroacetic acid or methane sulfonic acid to form the amine (7).

20. The process of claim 19, wherein the PG-protected amine (6A) is prepared by
coupling an aryl fluoride of Formula (4A) 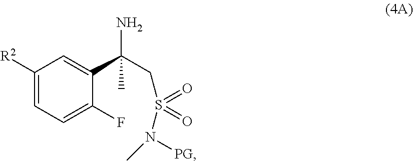
or a salt thereof, wherein R² is:
halo, selected from the group consisting of bromo, chloro and iodo;
a group of the formula —O—S(O)₂—R^{2a}, wherein R^{2a} is methyl, chloromethyl, dichloromethyl, phenyl, p-trifluoromethylbenzyl, p-toluenyl, p-bromophenyl, p-fluorophenyl, p-methoxyphenyl, 2-nitrophenyl, 4-nitrophenyl, and 2,4-dichlorophenyl; or
a diazonium group; and
PG is a protecting group;
with 5-fluoropicolinamide,
in the presence of:
a copper or palladium reagent;
a ligand; and
a Brønsted base to form the PG-protected amine (6A).

21. The process of claim 19, wherein the aryl fluoride of Formula (4A) is in the form of the (−)-O,O-dibenzoyl-L-tartrate salt and PG is PMB.

22. The process of claim 21, wherein the aryl fluoride (4A) is prepared by treating a methylsulfonamide 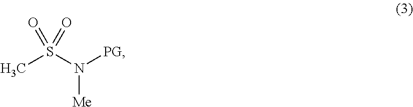
wherein PG is a protecting group, with an alkali metal base to form the alkali metalate species of the methylsulfonamide (3); reacting the alkali metalate species of the methylsulfonamide (3) with a sulfinyl imine 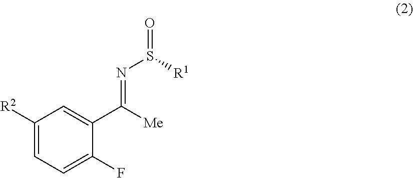
to form an aryl fluoride (4) 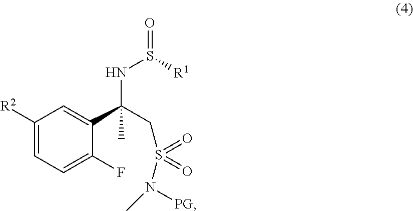
wherein R₁ is C₁-C₆ alkyl or phenyl, wherein the phenyl is unsubstituted or substituted by 1 to 3 substituents selected from the group consisting of C₁-C₄ alkyl, C₁-C₄ haloalkyl, —O—C₁-C₄ alkyl, —O—C₁-C₄ haloalkyl, halo, and nitro; and wherein R² is halo, selected from the group consisting of bromo, chloro and iodo; a group of the formula —O—S(O)₂—R^{2a}, wherein R^{2a} is methyl, chloromethyl, dichloromethyl, phenyl, p-trifluoromethylbenzyl, p-toluenyl, p-bromophenyl, p-fluorophenyl, p-methoxyphenyl, 2-nitrophenyl, 4-nitrophenyl, and 2,4-dichlorophenyl; and a diazonium group;
then further treating said aryl fluoride (4) with a strong acid to form (4A).

23. A compound according to the Formula (7):

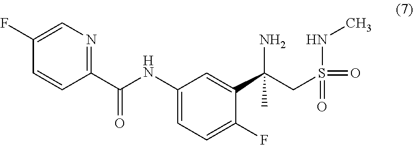 [SMILES: CNS(=O)(=O)C[C@](C)(N)c1cc(NC(=O)c2ccc(F)cn2)ccc1F]

24. A compound according to the Formula (6): 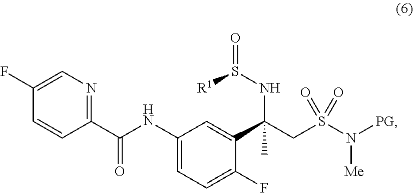
or a salt thereof, wherein:
R₁ is C₁-C₆ alkyl; or phenyl, wherein the phenyl is unsubstituted or substituted by 1 to 3 substituents selected from the group consisting of C₁-C₄ alkyl, C₁-C₄ haloalkyl, —O—C₁-C₄ alkyl, —O—C₁-C₄ haloalkyl, halo, and nitro; and PG is a protecting group.

25. The compound of claim 21, wherein R¹ is tert-butyl and PG is PMB.

26. A compound according to Formula (6A): 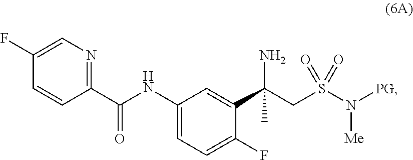
wherein PG is a protecting group.

27. A salt according to Formula (7A): 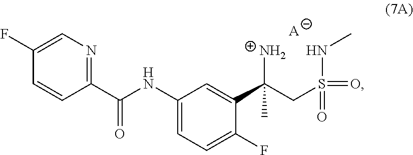
wherein A^{\textcircled{-}} is a salt of: L-tartaric acid, L-(+)-mandelic acid, L-(−)-malic acid, (1S)-(+)-10-camphorsulfonic acid, (−)-di-O,O-p-toluyl-L-tartaric acid, (−)-O,O-dibenzoyl-L-tartaric acid, (+)-camphoric acid, L-pyroglutamic acid, (1S)-(−)-camphanic acid, L-valine, (1S)-(+)-3-bromocamphor-10-sulfonic acid hydrate, L-histidine, D-tartaric acid, D-(−)-mandelic acid, D-(+)-malic acid, (1R)-(−)-10-camphorsulfonic acid, (+)-Di-O,O-p-toluyl-D-tartaric acid, (+)-O,O-dibenzoyl-D-tartaric acid, (−)-camphoric acid, D-pyroglutamic acid, (1R)-(+)-camphanic acid, D-valine, (+)-naproxen, or L-isoleucine.

28. A compound of Formula (4): 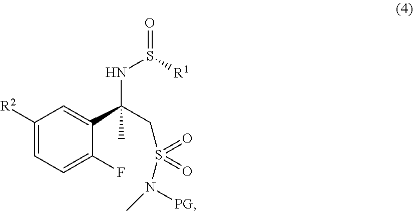
wherein
R₁ is:
C₁-C₆ alkyl; or
phenyl, wherein the phenyl is unsubstituted or substituted by 1 to 3 substituents selected from the group consisting of C₁-C₄ alkyl, C₁-C₄ haloalkyl, —O—C₁-C₄ alkyl, —O—C₁-C₄ haloalkyl, halo, and nitro;
R² is:
halo, selected from the group consisting of bromo, chloro and iodo;
a group of the formula —O—S(O)₂—R^{2a}, wherein R^{2a} is methyl, chloromethyl, dichloromethyl, phenyl, p-trifluoromethylbenzyl, p-toluenyl, p-bromophenyl, p-fluorophenyl, p-methoxyphenyl, 2-nitrophenyl, 4-nitrophenyl, and 2,4-dichlorophenyl; or
a diazonium group; and
PG is a protecting group.

29. A compound of Formula (4A): 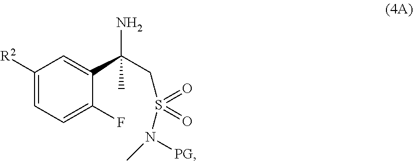
wherein
R² is:
halo, selected from the group consisting of bromo, chloro and iodo;
a group of the formula —O—S(O)₂—R^{2a}, wherein R^{2a} is methyl, chloromethyl, dichloromethyl, phenyl, p-trifluoromethylbenzyl, p-toluenyl, p-bromophenyl, p-fluorophenyl, p-methoxyphenyl, 2-nitrophenyl, 4-nitrophenyl, and 2,4-dichlorophenyl; or
a diazonium group; and
PG is a protecting group.

30. A compound of Formula (8): 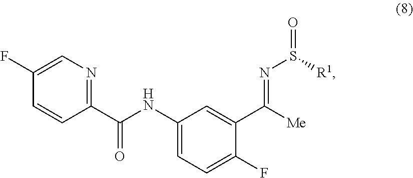
wherein R₁ is C₁-C₆ alkyl; or phenyl, wherein the phenyl is unsubstituted or substituted by 1 to 3 substituents independently selected from the group consisting of C₁-C₄ alkyl, C₁-C₄ haloalkyl, —O—C₁-C₄ alkyl, —O—C₁-C₄ haloalkyl, halo, and nitro.

31. A compound of Formula (5):

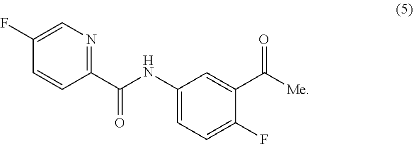 [SMILES: CC(=O)c1cc(NC(=O)c2ccc(F)cn2)ccc1F]

32. A compound of Formula (9): 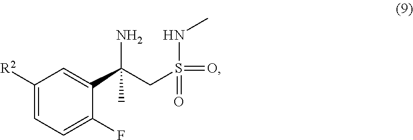
wherein R² is:
a group of the formula —O—S(O)₂—R^{2a}, wherein R^{2a} is methyl, chloromethyl, dichloromethyl, phenyl, p-trifluoromethylbenzyl, p-toluenyl, p-bromophenyl, p-fluorophenyl, p-methoxyphenyl, 2-nitrophenyl, 4-nitrophenyl, and 2,4-dichlorophenyl; or
a diazonium group.
